(19) United States
(12) Patent Application Publication
(10) Pub. No.: US 2020/0061124 A1
(43) Pub. Date: Feb. 27, 2020

(54) METHODS FOR MAKING AND USING DEDIFFERENTIATED AND STEM-LIKE HUMAN CELLS

(71) Applicant: The Regents of the University of California, Oakland, CA (US)

(72) Inventors: Joseph WAWRZYNIAK, La Jolla, CA (US); David CHERESH, La Jolla, CA (US)

(21) Appl. No.: 16/467,365

(22) PCT Filed: Dec. 1, 2017
(86) PCT No.: PCT/US2017/064250

§ 371 Date: Jun. 6, 2019

Related U.S. Application Data
(60) Provisional application No. 62/430,777, filed on Dec. 6, 2016.

(51) Int. Cl. A61K 35/545; C12N 5/074

(52) U.S. Cl. CPC A61K 35/545; C12N 5/0696; C12N 2501/585; C12N 2501/603; C12N 2510/00; C12N 2501/605; C12N 2506/28; C12N 2506/45; C12N 2501/604

(57) ABSTRACT

Provided are methods for making highly dedifferentiated and stem-like human cells from human umbilical vein endothelial cells (HUVECs) ectopically expressing integrin β3. Also provided are methods for making ectoderm, mesoderm, and endoderm cells from HUVECs ectopically expressing integrin β3. Also provided are methods for making neural cells, or cells having neuronal-like morphology, from HUVECs ectopically expressing integrin β3. Provided are methods for making cardiomyocytes, or cells having cardiomyocyte-like morphology, from HUVECs ectopically expressing integrin β3. Provided are methods for the production of pluripotent stem cells comprising expressing integrin β3 in primary human endothelial cells. In alternative embodiments, provided are methods for inducing αvβ3 clustering, and to accelerate or facilitate angiogenesis, tissue remodeling or repair, or wound healing, for example, to accelerate healing after an infarction.

### RELATED APPLICATIONS

This application claims the benefit of priority to U.S. Provisional Patent Application Ser. No. 62/430,777, Dec. 6, 2016. The aforementioned application is expressly incorporated herein by reference in its entirety and for all purposes.

### STATEMENT AS TO FEDERALLY SPONSORED RESEARCH

This invention was made with government support under National Institutes of Health (NIH) grant no. T32 CA121938. The government has certain rights in the invention.

### TECHNICAL FIELD

This invention generally relates to medicine and drug screening. In alternative embodiments, provided are methods for making highly dedifferentiated and stem-like human cells from human umbilical vein endothelial cells (HUVECs) ectopically expressing integrin β3 (or ITGB3). In alternative embodiments, provided are methods for reprogramming endothelial cells into a dedifferentiated state and creating an induced pluripotent stem cell (iPSCs) by inducing αvβ3 clustering. In alternative embodiments, provided are methods for inducing αvβ3 clustering, and to accelerate or facilitate angiogenesis, tissue remodeling or repair, or wound healing, for example, to accelerate healing after an infarction.

### BACKGROUND

The standardized approach to reprogram somatic cells requires the introduction of 4 genes known as the Yamanaka factors (Oct-4, Sox-2, Klf4 and c-Myc) into target cells using retroviruses. Following stable introduction of the Yamanaka factors into somatic cells (6 days), cellular reprogramming to induced pluripotent stem cells (iPSCs) takes approximately 30 days. Alternatively, the Yamanaka factors (Oct-4, Sox-2, Klf4 and c-Myc) and NANOG are master transcription factors associates with pluripotency, although expression of the Yamanaka factors is sufficient to drive pluripotency. Expression of the Yamanaka factors is associated with a more aggressive cancer phenotype, including more self-renewal, tumor initiation, anchorage-independence and drug resistance.

Integrin β3 (or ITGB3, also called CD61) is expressed on angiogenic endothelial cells and invasive tumor cells, and ligands binding to ITGB3 drives diverse cell signaling pathways. ITGB3 also can promote cell anchorage-independence, and can contribute to oncogenicity and metastatic potential and pregnancy-associated breast cancer. ITGB3 is enriched in metastatic cells, and may be involved in cancer cell remodeling by driving stemness and drug resistance through an αvβ3-KRAS-RalB complex. The integrin αvβ3 is preferentially expressed on tumor cell blood vessels. Tumor angiogenesis leads to chronic vascular remodeling.

Integrin β3 expression is absent on terminally differentiated endothelial cells (ECs) in vivo; integrin β3 is absent in quiescence. Angiogenic blood vessels in tumors or during development are highly positive for integrin β3 expression. Integrin β3 is highly expressed on proliferating ECs growing in full serum. Thick basement membrane, a terminal signal, initiates EC differentiation and the cells become αvβ3 (avb3) negative.

### SUMMARY

In alternative embodiments, provided are methods for:
upregulating pluripotency genes in a somatic cell, or a human endothelial cell,
dedifferentiating the somatic cell or human endothelial cell to become a stem-like cell, or reprogramming the somatic cell or endothelial cell into a dedifferentiated state to generate an induced pluripotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair, and/or
enhancing wound repair,

comprising ectopically expressing integrin β3 in the somatic cell or the human endothelial cell, or expressing or overexpressing in the somatic cell or the human endothelial cell a heterologous integrin β3,

wherein optionally the human endothelial cell is a human umbilical vein endothelial cell (HUVEC),

and optionally the pluripotency gene is a NANOG, OCT4, SOX2, and/or KLF4 gene,

and optionally the dedifferentiated or reprogrammed human endothelial cell loses its endothelial identity, optionally comprising loss of CD31, VWF, VE-cadherin, and VEGFR2, and gain of pluripotency markers such as NANOG, OCT4, SOX2, and KLF4,

and optionally ectopic expression of the integrin β3 in the somatic cell or the human endothelial cell is by transduction of a vector or a virus, optionally an adenovirus or a lentivirus (having contained therein an integrin β3-expressing nucleic acid), and expressing the integrin β3.

In alternative embodiments, the methods further comprise inducing the dedifferentiated or reprogrammed human somatic or endothelial cell, or the induced pluripotent stem cell (iPSC), to express lineages markers associated with all three germ layers ectoderm, mesoderm and endoderm, and/or inducing the dedifferentiated human somatic or endothelial cell to differentiate to one or all three germ layers ectoderm, mesoderm and endoderm, optionally comprising placing or incubating the dedifferentiated human somatic or endothelial cell in spheroid forming conditions.

In alternative embodiments, provided are methods for:
upregulating pluripotency genes in an αvβ3-expressing human somatic cell, an αvβ3-endothelial cell, or an αvβ3-expressing human endothelial cell,
dedifferentiating an αvβ3-expressing human somatic cell, an αvβ3-endothelial cell, or an αvβ3-expressing human endothelial cell to become a stem-like cell,
reprogramming an αvβ3-expressing human somatic cell, an αvβ3-endothelial cell, or an αvβ3-expressing human endothelial cell into a dedifferentiated state to generate an induced pluripotent or multipotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair,
enhancing wound repair, and/or
enhancing or accelerating healing, tissue healing or remodeling, vascularization or revascularization and/or tissue repair after an ischemic event, a tissue injury, a wound (e.g., surgical or traumatic), a burn, or an infarction,
wherein optionally the infarction is a myocardial infarction (MI) or a brain infarction or a stroke,
and optionally the ischemic event or injury is caused by an occlusion, an embolism or a trauma, or an aneurysm,
and optionally the ischemic event, wound or tissue injury is or is caused by: a diabetic ulcer, a corneal ischemic event, a stroke, a myocardial infarction, a mitral valve disease, a chronic atrial fibrillation, a cardiomyopathy, a prosthesis,
and optionally the tissue healing or remodeling comprises healing or remodeling of skin (including epidermal or dermal tissues), connective tissue, fascia, bone, cartilage, tendon or muscle,
and optionally the vascularization or revascularization, tissue healing or remodeling comprises healing or remodeling or the treatment of: retinal ischemia, diabetic retinopathy, or ocular ischemic syndrome (OIS), cardiac ischemia, bowel ischemia or ischemic colitis, brain ischemia, limb ischemia, or cutaneous ischemia, hypotension, sickle cell disease, arteriovenous malformations or peripheral artery occlusive disease,
and optionally the tissue healing or remodeling comprises healing or remodeling trauma or injury due to radiotherapy,

comprising: clustering of cell surface αvβ3,

and optionally the clustering of cell surface αvβ3 is by use of a multivalent ligand that binds to either integrin αv, integrin β3 or integrin αvβ3 (exposure of the cell surface to the multivalent ligand),

and optionally the clustering of cell surface αvβ3 is by (comprises) use of, or the clustering of cell surface αvβ3 comprises administration to an individual in need thereof:
an antibody or antibodies that can bind integrin αvβ3 and cluster αvβ3 on the cell surface, or a first antibody that can bind integrin αvβ3 and a second antibody that can bind to the first antibody such that the antibody binding clusters αvβ3 on the cell surface;
a multivalent (greater than bi-valent) compound capable of clustering cell surface αvβ3, wherein optionally the multivalent compound is a pentavalent molecule,
   and optionally the multivalent compound capable of clustering αvβ3 on a cell surface comprises:
(a) an extracellular matrix (ECM) protein, or an ECM homogenate or ECM-derived composition, capable of clustering αvβ3 on a cell surface,
and optionally the ECM comprises vitronectin, fibrinogen, and/or fibronectin,
and optionally the ECM comprises a decellularized ECM matrix or an ECM matrix hydrogel, optionally a myocardial matrix or a myocardial matrix hydrogel,
(b) a polysaccharide or glycosylated polypeptide capable of clustering αvβ3 on a cell surface,
(c) a lectin, a lectin capable of specifically binding of β-galactosides, or a Galectin-3 or a Galectin-9, capable of clustering αvβ3 on a cell surface;
(d) a compound comprising three or more RGD peptides (a αvβ3 binding motif) or mimetic RGD peptides capable of clustering αvβ3 on a cell surface, wherein optionally the compound comprises a polypeptide or a hydrogel;
(e) manganese cations (Mn²⁺) or a composition comprising a plurality of manganese cations (Mn²⁺), capable of clustering αvβ3 on a cell surface;
(f) a viral coat protein, or a composition comprising a plurality of viral coat proteins, a capsid or a virion that can cluster cell surface αvβ3,
(g) any combination of (a) to (f), or equivalents thereof,
thereby:
   upregulating pluripotency genes in an αvβ3-expressing human somatic cell, an αvβ3-endothelial cell, or an αvβ3-expressing human endothelial cell,
   dedifferentiating an αvβ3-expressing human somatic cell, an αvβ3-endothelial cell, or an αvβ3-expressing human endothelial cell to become a stem-like cell,
   reprogramming an αvβ3-expressing human somatic cell, an αvβ3-endothelial cell, or an αvβ3-expressing human endothelial cell into a dedifferentiated state to generate an induced pluripotent or multipotent stem cell (iPSC),
   increasing endothelial cell plasticity during angiogenesis,
   increasing or enhancing vascularization and/or angiogenesis,
   promoting or initiating endothelial remodeling,
   increasing, facilitating or enhancing tissue remodeling or repair,
   enhancing wound repair, and/or
   enhancing or accelerating healing, tissue healing or remodeling, vascularization and/or tissue repair after an ischemic event, a tissue injury, a wound, a burn, or an infarction.

In alternative embodiments of the methods, the antibody or antibodies that can bind integrin αvβ3 and cluster αvβ3 on the cell surface, or the multivalent compound capable of clustering cell surface αvβ3, is/are:
formulated as a pharmaceutical composition,
formulated with a pharmaceutically acceptable excipient,
administered directly to, into, locally to, or adjacent to, a wound or injury site or tissue, a site or tissue requiring increased or enhanced vascularization and/or angiogenesis, a site or tissue needing promotion or initiation of endothelial remodeling, an infarction site, to an injured or infarcted heart or other tissue or organ, or to any tissue or organ in need of increased or enhanced vascularization or tissue repair,
wherein optionally the administration is by injection or by placement of an implant.

In alternative embodiments, the methods further comprise inducing the dedifferentiated or reprogrammed human somatic or endothelial cell, or the induced pluripotent stem cell (iPSC), to express lineages markers associated with all three germ layers ectoderm, mesoderm and endoderm, and/or inducing the dedifferentiated human somatic or endothelial cell to differentiate to one or all three germ layers ectoderm, mesoderm and endoderm, optionally comprising placing or incubating the dedifferentiated human somatic or endothelial cell in spheroid forming conditions.

In alternative embodiments, provided are method for:
upregulating pluripotency genes in a αvβ3-expressing human somatic cell or human endothelial cell, and/or
dedifferentiating the αvβ3-expressing human somatic cell or human endothelial cell to become a stem-like cell, or reprogramming the human somatic cell or endothelial cell into a dedifferentiated state to generate an induced pluripotent stem cell (iPSC),

comprising ectopically expressing integrin 133 (HUVEC 133⁺) or expressing in the human somatic cell or the human endothelial cell a heterologous integrin 133 (HUVEC 133⁺) to generate a conditioned or altered media, and culturing or exposing the human somatic cell or the human endothelial cell to the conditioned or altered media,

wherein optionally the human endothelial cell is a human umbilical vein endothelial cell (HUVEC),

and optionally the pluripotency gene is a NANOG, OCT4, SOX2, and/or KLF4 gene,

and optionally the dedifferentiated or reprogrammed human endothelial cell loses its endothelial identity, optionally comprising loss of CD31, VWF, VE-cadherin, and VEGFR2, and gain of pluripotency markers such as NANOG, OCT4, SOX2, and KLF4,

and optionally ectopic expression of the integrin 133 (HUVEC 133⁺) in the human somatic cell or the human endothelial cell is by transduction of a vector or a virus, optionally a lentivirus, expressing the integrin β3 (having contained therein integrin 133 (HUVEC 133⁺)-expressing nucleic acid).

In alternative embodiments, the methods further comprise inducing the dedifferentiated or reprogrammed human somatic or endothelial cell, or the induced pluripotent stem cell (iPSC), to express lineages markers associated with all three germ layers ectoderm, mesoderm and endoderm, and/or inducing the dedifferentiated human somatic or endothelial cell to differentiate to one or all three germ layers ectoderm, mesoderm and endoderm, optionally comprising placing or incubating the dedifferentiated human somatic or endothelial cell in spheroid forming conditions.

In alternative embodiments, provided are Uses of a human somatic cell or a human endothelial cell ectopically expressing an integrin β3 or expressing a heterologous integrin β3 to:
upregulate pluripotency genes in a human somatic cell, or a human endothelial cell, and/or
dedifferentiate the human somatic cell or human endothelial cell to become a stem-like cell, or reprogram the human somatic cell or human endothelial cell into a dedifferentiated state to generate an induced pluripotent stem cell (iPSC).

In alternative embodiments, provided are Uses of a human somatic cell or a human endothelial cell ectopically expressing an integrin 133 (HUVEC 133⁺) or expressing a heterologous integrin 133 (HUVEC 133⁺) to
upregulate pluripotency genes in the human somatic cell, or the human endothelial cell, and/or
dedifferentiate the human somatic cell or human endothelial cell to become a stem-like cell, or reprogram the human somatic cell or human endothelial cell into a dedifferentiated state to generate an induced pluripotent stem cell (iPSC).

In alternative embodiments, provided are compositions capable of clustering cell surface αvβ3, optionally a multivalent ligand that binds to either integrin αv, integrin β3 or integrin αvβ3 and is capable of clustering cell surface αvβ3, for use in:
upregulating pluripotency genes in an αvβ3-expressing human somatic cell, an αvβ3-endothelial cell, or an αvβ3-expressing human endothelial cell,
dedifferentiating an αvβ3-expressing human somatic cell, an αvβ3-endothelial cell, or an αvβ3-expressing human endothelial cell to become a stem-like cell,
reprogramming an αvβ3-expressing human somatic cell, an αvβ3-endothelial cell, or an αvβ3-expressing human endothelial cell into a dedifferentiated state to generate an induced pluripotent or multipotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair,
enhancing wound repair, and/or
enhancing or accelerating healing, tissue healing or remodeling, vascularization or revascularization and/or tissue repair after an ischemic event, a tissue injury, a wound (e.g., surgical or traumatic), a burn, or an infarction,
wherein optionally the infarction is a myocardial infarction (MI) or a brain infarction or a stroke,
and optionally the ischemic event or injury is caused by an occlusion, an embolism or a trauma, or an aneurysm,
and optionally the ischemic event, wound or tissue injury is or is caused by: a diabetic ulcer, a corneal ischemic event, a stroke, a myocardial infarction, a mitral valve disease, a chronic atrial fibrillation, a cardiomyopathy, a prosthesis,
and optionally the tissue healing or remodeling comprises healing or remodeling of skin (including epidermal or dermal tissues), connective tissue, fascia, bone, cartilage, tendon or muscle,
and optionally the vascularization or revascularization, tissue healing or remodeling comprises healing or remodeling or the treatment of: retinal ischemia, diabetic retinopathy, or ocular ischemic syndrome (OIS), cardiac ischemia, bowel ischemia or ischemic colitis, brain ischemia, limb ischemia, or cutaneous ischemia, hypotension, sickle cell disease, arteriovenous malformations or peripheral artery occlusive disease,
and optionally the tissue healing or remodeling comprises healing or remodeling trauma or injury due to radiotherapy,

and optionally the clustering of cell surface αvβ3 is by (comprises) use of, or the clustering of cell surface αvβ3 comprises administration to an individual in need thereof:
an antibody or antibodies that can bind integrin αvβ3 and cluster αvβ3 on the cell surface, or a first antibody that can bind integrin αvβ3 and a second antibody that can bind to the first antibody such that the antibody binding clusters αvβ3 on the cell surface;
a multivalent (greater than bi-valent) compound capable of clustering cell surface αvβ3, wherein optionally the multivalent compound is a pentavalent molecule,
   and optionally the multivalent compound capable of clustering αvβ3 on a cell surface comprises:
(a) an extracellular matrix (ECM) protein, or an ECM homogenate or ECM-derived composition, capable of clustering αvβ3 on a cell surface,
and optionally the ECM comprises vitronectin, fibrinogen, and/or fibronectin,
and optionally the ECM comprises a decellularized ECM matrix or an ECM matrix hydrogel, optionally a myocardial matrix or a myocardial matrix hydrogel,
(b) a polysaccharide or glycosylated polypeptide capable of clustering αvβ3 on a cell surface,
(c) a lectin, a lectin capable of specifically binding of β-galactosides, or a Galectin-3 or a Galectin-9, capable of clustering αvβ3 on a cell surface;
(d) a compound comprising three or more RGD peptides (a αvβ3 binding motif) or mimetic RGD peptides capable of clustering αvβ3 on a cell surface, wherein optionally the compound comprises a polypeptide or a hydrogel;
(e) manganese cations (Mn²⁺) or a composition comprising a plurality of manganese cations (Mn²⁺), capable of clustering αvβ3 on a cell surface;
(f) a viral coat protein, or a composition comprising a plurality of viral coat proteins, a capsid or a virion that can cluster cell surface αvβ3,
(g) any combination of (a) to (f), or equivalents thereof.

In alternative embodiments, provided are Uses of a composition capable of clustering cell surface αvβ3, optionally use of a multivalent ligand that binds to either integrin αv, integrin β3 or integrin αvβ3 and is capable of clustering cell surface αvβ3, in the manufacture of a medicament for:
upregulating pluripotency genes in an αvβ3-expressing human somatic cell, an αvβ3-endothelial cell, or an αvβ3-expressing human endothelial cell,
dedifferentiating an αvβ3-expressing human somatic cell, an αvβ3-endothelial cell, or an αvβ3-expressing human endothelial cell to become a stem-like cell,
reprogramming an αvβ3-expressing human somatic cell, an αvβ3-endothelial cell, or an αvβ3-expressing human endothelial cell into a dedifferentiated state to generate an induced pluripotent or multipotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair,
enhancing wound repair, and/or
enhancing or accelerating healing, tissue healing or remodeling, vascularization or revascularization and/or tissue repair after an ischemic event, a tissue injury, a wound (e.g., surgical or traumatic), a burn, or an infarction,
wherein optionally the infarction is a myocardial infarction (MI) or a brain infarction or a stroke,
and optionally the ischemic event or injury is caused by an occlusion, an embolism or a trauma, or an aneurysm,
and optionally the ischemic event, wound or tissue injury is or is caused by: a diabetic ulcer, a corneal ischemic event, a stroke, a myocardial infarction, a mitral valve disease, a chronic atrial fibrillation, a cardiomyopathy, a prosthesis,
and optionally the tissue healing or remodeling comprises healing or remodeling of skin (including epidermal or dermal tissues), connective tissue, fascia, bone, cartilage, tendon or muscle,
and optionally the vascularization or revascularization, tissue healing or remodeling comprises healing or remodeling or the treatment of: retinal ischemia, diabetic retinopathy, or ocular ischemic syndrome (OIS), cardiac ischemia, bowel ischemia or ischemic colitis, brain ischemia, limb ischemia, or cutaneous ischemia, hypotension, sickle cell disease, arteriovenous malformations or peripheral artery occlusive disease,
and optionally the tissue healing or remodeling comprises healing or remodeling trauma or injury due to radiotherapy,

and optionally the clustering of cell surface αvβ3 is by (comprises) use of, or the clustering of cell surface αvβ3 comprises administration to an individual in need thereof:
an antibody or antibodies that can bind integrin αvβ3 and cluster αvβ3 on the cell surface, or a first antibody that can bind integrin αvβ3 and a second antibody that can bind to the first antibody such that the antibody binding clusters αvβ3 on the cell surface;
a multivalent (greater than bi-valent) compound capable of clustering cell surface αvβ3, wherein optionally the multivalent compound is a pentavalent molecule,
   and optionally the multivalent compound capable of clustering αvβ3 on a cell surface comprises:
(a) an extracellular matrix (ECM) protein, or an ECM homogenate or ECM-derived composition, capable of clustering αvβ3 on a cell surface,
and optionally the ECM comprises vitronectin, fibrinogen, and/or fibronectin,
and optionally the ECM comprises a decellularized ECM matrix or an ECM matrix hydrogel, optionally a myocardial matrix or a myocardial matrix hydrogel,
(b) a polysaccharide or glycosylated polypeptide capable of clustering αvβ3 on a cell surface,
(c) a lectin, a lectin capable of specifically binding of β-galactosides, or a Galectin-3 or a Galectin-9, capable of clustering αvβ3 on a cell surface;
(d) a compound comprising three or more RGD peptides (a αvβ3 binding motif) or mimetic RGD peptides capable of clustering αvβ3 on a cell surface, wherein optionally the compound comprises a polypeptide or a hydrogel;
(e) manganese cations (Mn²⁺) or a composition comprising a plurality of manganese cations (Mn²⁺), capable of clustering αvβ3 on a cell surface;
(f) a viral coat protein, or a composition comprising a plurality of viral coat proteins, a capsid or a virion that can cluster cell surface αvβ3,
(g) any combination of (a) to (f), or equivalents thereof.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1 graphically illustrates mRNA levels (in “fold” levels) of integrin β3 (or ITGB3), integrin β5 (or ITGB5), and the Yamanaka factors Oct-4, Sox-2, Klf4 and c-Myc in human umbilical vein endothelial cells (HUVECs) ectopically expressing integrin β3, as compared to HUVECs with integrin β3 knockdown (i.e., expressing no integrin β3).

FIG. 2 illustrates an image showing that HUVECs ectopically expressing integrin β3 (right panel), when grown on basement membrane extract—which normally drives terminal differentiation—do not terminally differentiate, as compared to HUVECs that do not express HUVECs ectopically (left panel).

FIG. 3 illustrates an image showing that HUVECs ectopically expressing integrin β3 (right panel) become anchorage independent, as compared to HUVECs that do not express HUVECs ectopically (left panel).

FIG. 4A graphically illustrates the increase in mRNA levels (in “fold” levels) of stem cell markers: integrin β3 (or ITGB3), integrin β5 (or ITGB5), and the Yamanaka factors Oct-4, Sox-2, Klf4 and c-Myc; and the decrease in endothelial cell (EC) markers CD31, VWF, VE-cadherin, and VEGFR2, in HUVECs ectopically expressing integrin β3.

FIG. 4B illustrates an image of the Western blots from which the mRNA data illustrated in FIG. 4A was generated.

FIG. 5 illustrates images showing that HUVECs ectopically expressing integrin β3 (right panel) in 2-dimensional cultures (2D) generate induced pluripotent stem cell-like (iPS-like) colonies, as compared to HUVECs not ectopically expressing integrin β3 (left panel). Formation of iPS cells can be determined by confirming: expression of NANOG and OCT4, differentiation toward a specific lineage, e.g., loss of EC markers or gain of lineage markers; whether individual colonies form spheroid formation on poly-hema plates; whether the cells have specialized function; or, whether teratomas are formed.

FIG. 6 illustrates an image showing that ectopic expression of integrin β3 (middle and right, upper and lower, panels), as compared to control vector (no integrin β3) (upper and lower left panel), transform NDHF-1 human fibroblasts, as indicated by staining.

FIG. 7 illustrates an image showing that HUVEC p-5 cells (5^{th} passage) ectopically expressing integrin β3 (right panel) form embryoid bodies, as compared to HUVEC p-5 cells not ectopically expressing integrin β3 (left panel); this in vitro assay is used to determine pluripotency, or spontaneous germline differentiation, and is a classic assay used to determine if iPS cells have the ability to differentiate into 3 germ layers in vitro.

FIG. 8 graphically illustrates the spontaneous induction of germline lineage markers in spheroids by showing the increase in mRNA (fold) as compared to control for: endoderm (left panel), showing an increase in the marker FOXA1; mesoderm (middle panel), showing an increase in the marker MYOSIN and NODAL; and, ectoderm (right panel), showing an increase in PAX6 and OTX2.

FIG. 9 illustrates images showing that HUVECs ectopically expressing integrin β3, initially in stem cell media (upper left panel), can be induced to differentiate to a neuroectodermal fate (e.g., to a neuron), using a “maturation protocol” of: (1) activin A and b-27 supplement for 24 hours; (2) activin A, b-27 supplement and BMP4 for the next five days; and (3) complete media with insulin for the next 2 to 3 weeks (upper right panel, in 6th day of protocol; day 7 of neural maturation protocol shown lower right and left panels).

FIG. 10 graphically illustrates mRNA expression (in fold) of markers expressed in day 12 cells of the HUVECs exposed to a “maturation protocol” as shown in FIG. 9, where increased expression of PAX6, OTX2 and PAX2, and decreased expression of NANOG, show a directed differentiation towards a neural-ectodermal fate, e.g., differentiation to a neuron.

FIG. 11 schematically illustrates a potential mechanism by which αvβ3 (avb3) clustering can reprogram a cancer cell to become a stem cell; as provided herein are methods for reprogramming endothelial cells into a dedifferentiated state and creating an induced pluripotent stem cell (iPSCs) by inducing αvβ3 clustering.

FIG. 12 illustrates images showing that in HUVECs ectopically expressing integrin β3, αvβ3 localizes with KRAS2B.

FIG. 13 illustrates images showing that in HUVECs ectopically expressing integrin β3, αvβ3 also localizes with Ras-related protein R-Ras (RRAS).

FIG. 14 graphically illustrates data (tumor volume in mm³ as a function of days post injection) showing that HUVECs ectopically expressing integrin β3 cause tumor formation in mice, where the study illustrated included 12 mice and 2×10⁶ cells to the left and right flanks of the mice, and tumor formation was seen in 6/6 of β3+ mice and 0/6 of β3 minus mice.

FIG. 15 illustrates images showing that HUVECs ectopically expressing integrin β3 have a change in morphology by day 12, where small compact colonies are formed (left panel), versus no change in morphology in day 12 negative control (right panel); ectopic expression of integrin β3 in HUVECs can convert these cells to pluripotent stem cells.

FIG. 16 illustrates images (left panel) showing that HUVECs ectopically expressing integrin β3 can lead to formation of iPS-like colonies in confluent HUVECs; where the right panel graphically illustrates the number of colonies induces by integrin β3 versus integrin β5 ectopic expression.

FIG. 17 illustrates images showing that αvβ3 is preferentially expressed on tumor vessels, the left panel showing normal tumor adjacent tissue and the right panel showing breast cancer tissue.

FIG. 18 schematically illustrates the reprogramming of the vasculature during angiogenesis, including the formation of the tip cell in response to angiogenic factors, where the tip cell displays new markers and invasive properties, and that stem gene such as NANOG and OCT4 are associated with vascular remodeling; noting that HUVECs ectopically expressing integrin β3 are induced to form iPS cells expressing NANOG and OCT4.

FIG. 19 graphically illustrates mRNA expression (in fold) of markers as compared to control after placing HUVECs ectopically expressing integrin β3 in spheroid forming conditions; the data demonstrates that all three germ-line markers: ectoderm (left panel), showing an increase in PAX6, OTX2 and PAX2; mesoderm (middle panel), showing an increase in the marker MYOSIN and NODAL; and, endoderm (right panel), showing an increase in the marker FOXA1, are spontaneously induced.

FIG. 20A illustrates images showing that HUVECs ectopically expressing integrin β3 (“HUVEC-β3s”) can be directed to differentiate to a neuronal cell fate, where the HUVEC-β3s were placed in neuronal cell differentiation media conditions for 22 days, and by day 12 the cells had a neuronal-like morphology.

FIG. 20B graphically illustrates mRNA expression (in fold) of markers as compared to control of the HUVEC-β3s of FIG. 20A, where the data shows by day 12 the HUVEC-β3s begin to express markers of early neuro-ectodermal differentiation, which losing expression of pluripotent stem genes NANOG and OCT4, and were expressing markers of mature neurons and astrocytes at both the mRNA level (FIG. 20B) and protein level (FIG. 20C).

FIG. 20C illustrates images of day 21 and 22 cells of FIG. 20B, showing that the differentiated cells were expressing markers of mature neurons and astrocytes at the protein level.

FIG. 21 graphically illustrates mRNA expression (in fold) of cardiomyocyte differentiation markers in HUVECs ectopically expressing integrin β3 (“HUVEC-β3 s”) after 14 days in cardiomyocyte differentiation conditions.

FIG. 22 illustrates an image of gels indicating the level of expression of stem cell markers NANOG and OCT4 (versus CD34 and CD133) in HUVECs ectopically expressing (or over-expressing) integrin β3 (“HUVEC-β3s”) (left panel), and graphically illustrates the level of mRNA expression (in fold) versus control of selected markers, including stem cell markers NANOG, SOX2, KLF4 and OCT4 (and as a positive control, integrin β3 (ITGB3), and as a negative control integrin β5 (ITGB5)), in the HUVEC-β3s; the data showing that ectopic expression of integrin β3 increases the expression of pluripotency genes in the HUVEC-β3s, and facilitates loss of endothelial identity, and increases expression of the pluripotency genes NANOG, SOX2, KLF4 and OCT4.

FIG. 23 graphically illustrates mRNA expression (in fold) in HUVECs after clustering of αvβ3 (avb3); where the data demonstrates that clustering of αvβ3 on the HUVECs drives stemness, as evidenced by increased expression of mRNA of the pluripotency genes NANOG, SOX2, KLF4 and OCT4.

FIG. 24A schematically illustrates the protocol of a study where Conditioned Media (CM) was collected from HUVECs ectopically expressing integrin β3 (“HUVEC β3”), and control HUVECs (not expressing HUVEC β3).

FIG. 24B graphically illustrates mRNA expression (in fold) in the HUVEC β3s, where the data demonstrates that ectopically expressed integrin β3 reprograms normal HUVEC to a dedifferentiated stem cell state; the HUVEC β3 CM (collected as illustrated in FIG. 24A) was cultured with normal HUVEC, and the CM induced the expression of the pluripotency genes OCT4, NANOG, SOX2, and KLF4, and decreased the expression of the endothelial markers CD31, VWF, CD34.

FIG. 25A illustrates images of HUVECs ectopically expressing integrin β3 (“HUVEC β3s”), where the HUVEC β3 show “stem-like” growth on: collagen I, showing colony formation (left panel); soft agar, showing anchorage independence (middle panel); and, Matrigel, showing colony formation (right panel).

FIG. 25B graphically illustrates gels demonstrating that there is a loss of expression of endothelial cell (EC) genes in HUVEC β3s (the endothelial markers CD31, VE-cad, and VEGFR2), and increase in expression of OCT4 and NANOG.

FIG. 26A graphically illustrates mRNA expression (in fold) in HUVEC β3s versus control cells (HUVEC, or EC, without integrin β3), where the cells are grown in low serum 0.1% (versus 10%), and where the data demonstrates that ectopically expressed integrin β3 includes expression of pluripotency markers OCT4, NANOG, SOX2, and TRA-1-60 in the low serum conditions.

FIG. 26B illustrates images of the cells of FIG. 25A, showing expression of the pluripotency markers OCT4, NANOG, SOX2, and TRA-1-60.

FIG. 27A-B illustrate how ectopic expression of integrin β3 in HUVEC (HUVEC β3s) increases the amount of integrin β3 on the HUVEC cell surface, and creates a new “window” of optimized surface expression that induces expression of pluripotency markers OCT4, NANOG, SOX2:

FIG. 27A illustrates cell sorting images of HUVEC control (no integrin β3), and HUVEC β3s, where five samples of cells were taken (cell sorted), where each sample had increasing amounts of integrin β3 cell surface expression (L1 the lowest level of expression of integrin β3, and L5 the highest level of expression of integrin β3); the data showing in increase amount of integrin β3 expressed on the HUVEC cell surface in the HUVEC β3s L4 and L5 samples, the L4 and L5 samples representing the ectopically expressed, versus the endogenous, integrin β3 (levels L1 to L3).

FIG. 27B graphically illustrates mRNA expression (in fold) in the sorted HUVEC β3s of FIG. 27A, where HUVEC β3s from the L4 sample express more of the pluripotency markers OCT4, NANOG, SOX2 than the L1, L2, L3 or L5 sorted samples.

FIG. 28 graphically illustrates mRNA expression (in fold) versus control, the data showing that the effect of clustering of αvβ3 on HUVECs can be reproduced with an optimal level of ectopic integrin β3 expression, where integrin β3 expression can continue to increase, but the level of expression of stem genes does not; instead, the level of expression of stem genes (the pluripotency markers OCT4, NANOG, SOX2) requires an optimal level of integrin β3 expression, as also illustrated in FIG. 27A-B.

FIG. 29A-C illustrate data demonstrating that extracellular matrix proteins (ECM), including ECM involved in wound healing, multivalently bind to integrin αvβ3 and induce the expression of pluripotent stem genes OCT4, NANOG, and SOX2:

FIG. 29A illustrates images of HUVECs grown in fibrinogen (FBN), left panel, and fibronectin (FN), right panel.

FIG. 29B illustrates images of HUVECs grown in denatured collagen (D-COLI), left panel, and vitronectin (VN), right panel.

FIG. 29C graphically illustrates mRNA expression (in fold) of pluripotent stem genes OCT4, NANOG, and SOX2 in the cells of FIG. 29A and FIG. 29B.

FIG. 30 graphically illustrates data showing that a tissue derived ECM hydrogel (a 3-dimensional, or 3-D, composite matrix, tissue-derived) that multivalently binds to integrin avb3 can induce the expression of the pluripotent stem genes OCT4, NANOG, and SOX2; where C is cardiac-derived ECM, L is lung-derived ECM, and S is skeletal-derived ECM.

FIG. 31A-B graphically illustrates data showing that stem cell induction on cardiac hydrogel is integrin avb3-dependent in two different endothelial cell populations HUVEC (FIG. 31A) and HCAEC (human coronary artery endothelial cells) (FIG. 31B): the data shows that function-blocking antibody against integrin avb3 prevents stem gene expression, while antibodies against integrin β5 or β1 do not prevent stem gene expression; in the study, 150,000 (150K) cells were seeded with or without antibodies in wells coated with Matrigel or cardiac ECM and incubated overnight (O/N) at 37° C., and RNA was collected 24 hours (hrs) later.

FIG. 32A-B illustrate how multivalent RGD molecules (as a penton base in adenovirus form at 0.5 μg/ml) cluster αvβ3 (as schematically illustrated in FIG. 32A), and after 24 hours, induce pluripotent stem gene expression in HUVECs, as indicated by the increase in expression of the pluripotent stem genes OCT4, NANOG, and SOX2, as compared to collagen (negative control) and vitronectin (as a positive control, as vitronectin is an αvβ3-clustering ECM); ITGB3 is the negative control, as graphically illustrated in FIG. 32B, where mRNA (fold) amounts versus collagen is shown; in the study, pentons were coated on a petri dish O/N at 4° C.; then 5% BSA was applied one hour at 37° C. to block non-specific binding, then cells were seeded at 150K, and RNA was collected 24 hrs later.

FIG. 33A-B illustrate data showing that soluble multivalent penton base (see FIG. 32A) binding to ECs induces stem genes OCT4, NANOG, and SOX2 (FIG. 33A), but the monovalent cilengitide (selective for αᵥ integrins) does not, this data demonstrating that multivalency in integrin avb3-ligation is required for induction of stem gene expression; in this study, wells were coated with Collagen I O/N at 4° C.; then 5% BSA was applied one hour at 37° C. to block non-specific binding, then cells were seeded at 200K in low serum media, and after cells adhere, or after about 30 minutes (min), penton base or cilengitide was added, then RNA was collected 24 hrs later.

FIG. 34 schematically illustrates an exemplary method for inducing αvβ3 clustering by using two antibodies: a first antibody that specifically binds to cell surface αvβ3, and a second antibody that specifically binds to the first antibody such that the binding induces clustering of the cell surface integrin αvβ3.

FIG. 35A-B graphically illustrate data showing that αvβ3 clustering, using the two-antibody model of FIG. 34, induces stem gene expression (OCT4, NANOG, SOX2 and KLF4), where both graphs show mRNA expression (fold) versus control, and FIG. 35A shows levels of stem cell markers, and FIG. 35B shows levels of endothelial cell (EC) markers; where the term “2nd” indicates the second antibody from FIG. 34 that specifically binds to the first antibody such that the binding induces clustering of the cell surface αvβ3, and the “αvβ3” indicates the first antibody that specifically binds to αvβ3, and “β1” indicates an antibody that binds to integrin β1.

FIG. 36A-B illustrate images showing that integrin αvβ3 is expressed in microvessels after myocardial ischemia and infarction; FIG. 36A illustrates an image of a heart, indicating the three zones shown in the histological section of FIG. 36B, which is stained for the endothelial marker CD31 and integrin αvβ3.

FIG. 37 illustrates images showing that integrin αvβ3 is expressed in the retinal vasculature during Oxygen-Induced Retinopathy (OIR) (left panel), as compared to control (no OIR, or “normal” control) (right panel), where integrin β3 is stained in red (as pointed to by the arrows in the left panel) and the endothelial marker CD31 is stained in green; in this study mouse pups were exposed to 75% O₂ from P7 to P12, harvested on P19.

FIG. 38A-B illustrate images showing that integrin αvβ3 is expressed in microvessels after focal cerebral ischemia; the images showing co-localization of integrin αvβ3 and fibrin in a large microvessel at 3 hours of MCA:O and one hour of reperfusion; FIG. 38A shows fibrin deposition using fluorescein isothiocyanate in a microvessel of 55 μm diameter, and FIG. 38B shows co-expression of integrin αvβ3 in the same microvessel; αvβ3 expression may reflect a response to fibrin formation and/or cytokine release as the first stage in vascular reorganization after focal cerebral ischemia.

FIG. 39 schematically illustrates an exemplary method for high-density clustering of integrin αvβ3 on cell surfaces to cause de-differentiation, resulting in loss of endothelial cell markers and gain of stem markers, thereby facilitating a stem-like reprogramming of endothelial cells, which increases endothelial cell plasticity during angiogenesis, which leads to increased or enhanced vascularization and tissue modeling and/or repair.

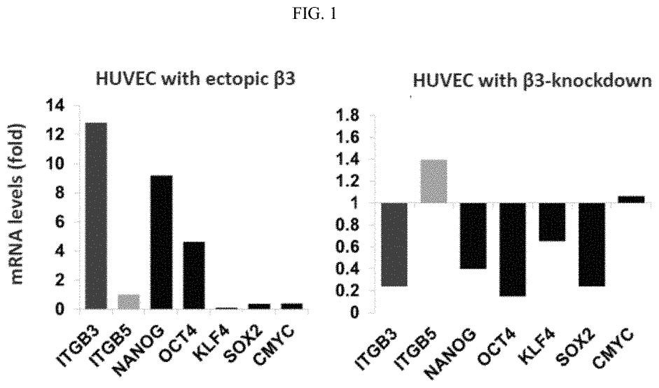

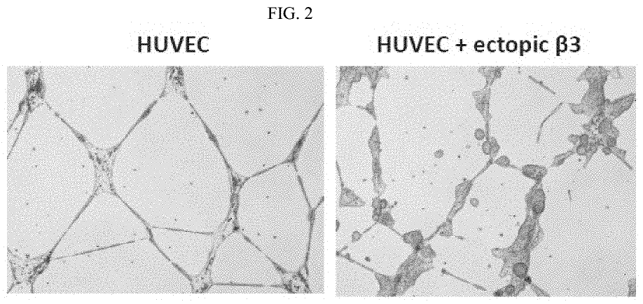

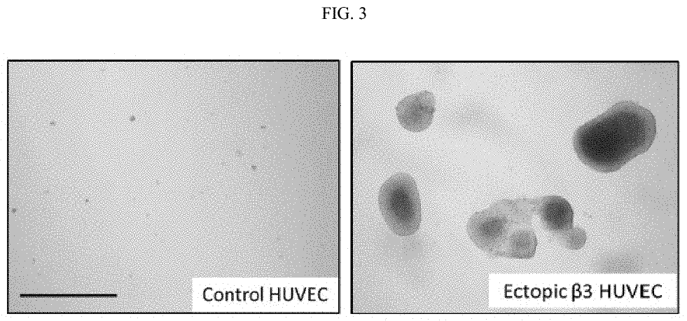

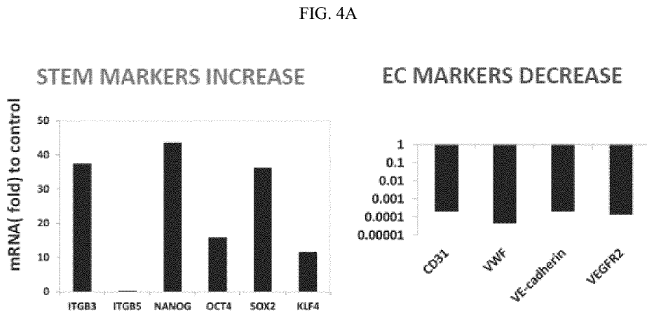

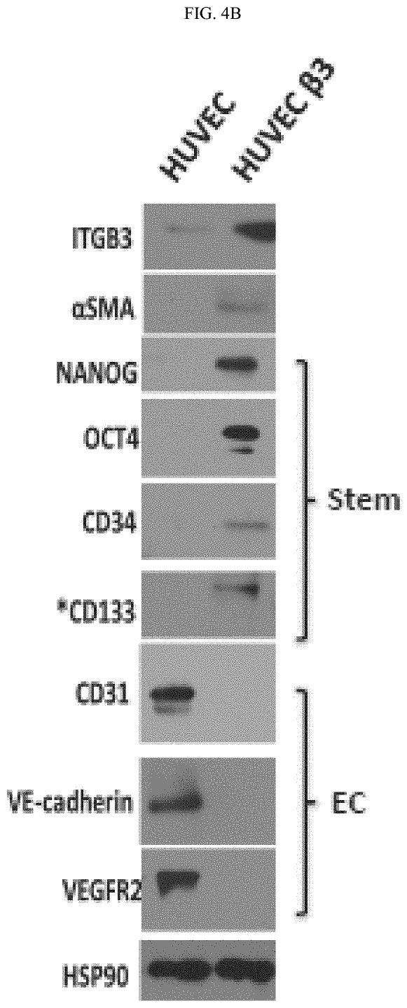

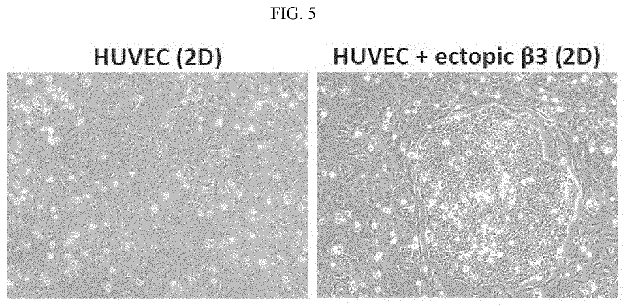

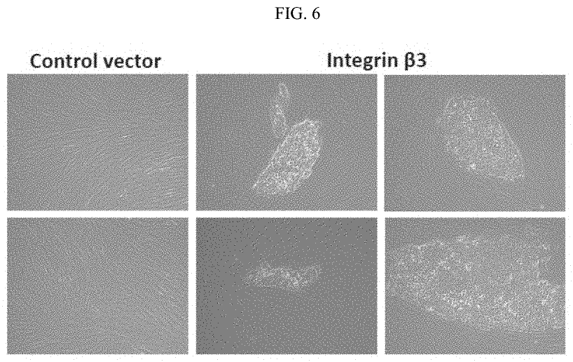

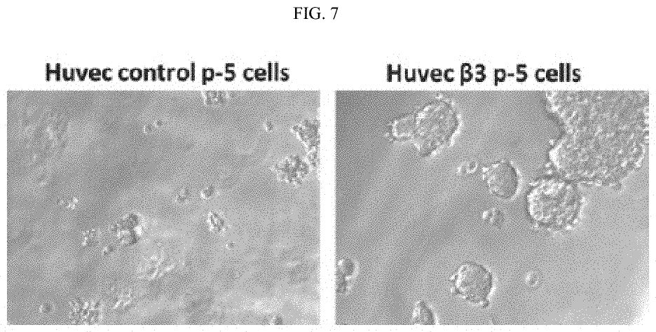

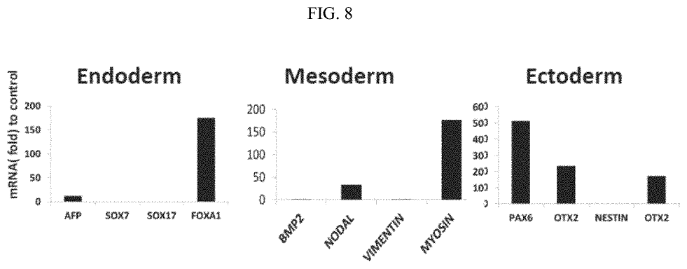

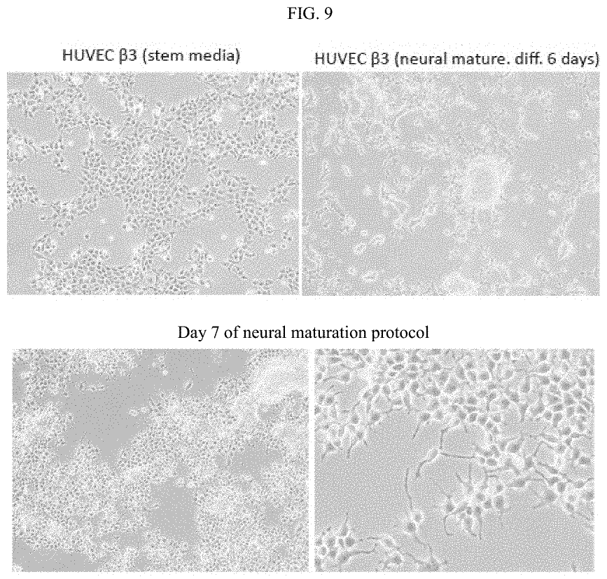

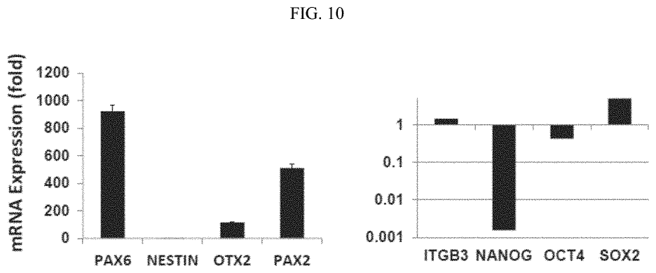

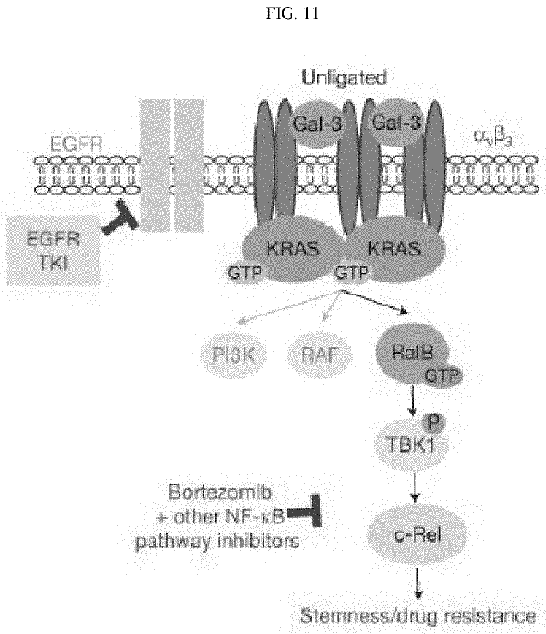

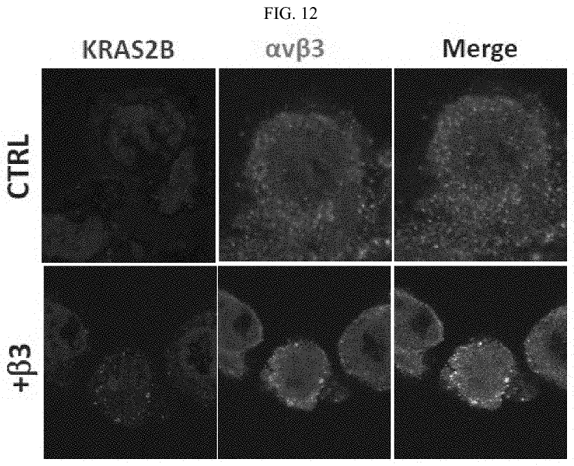

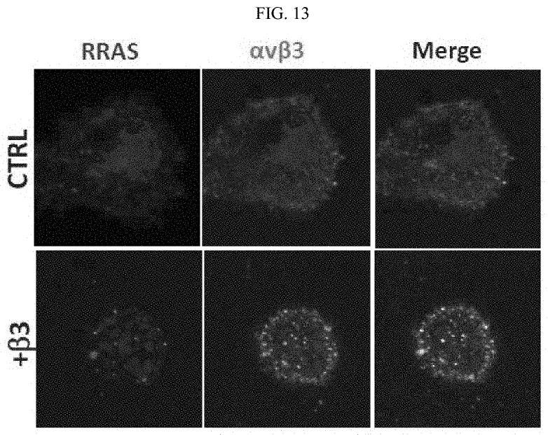

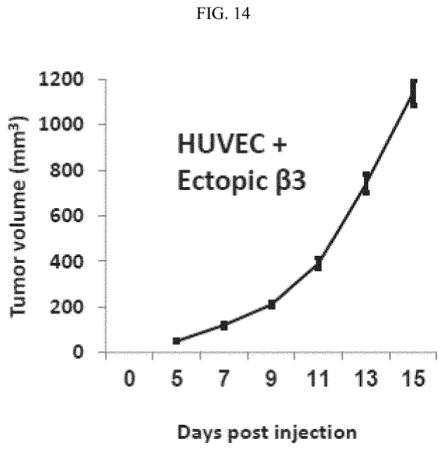

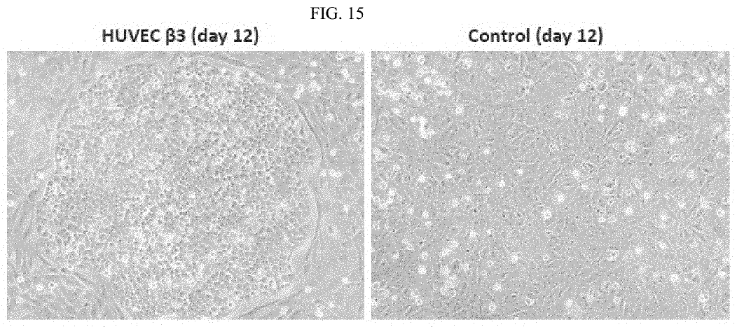

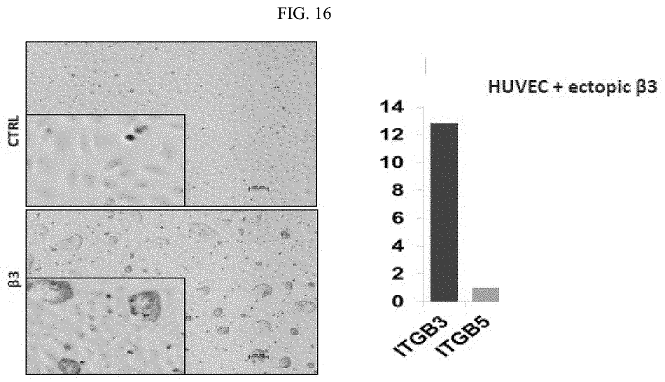

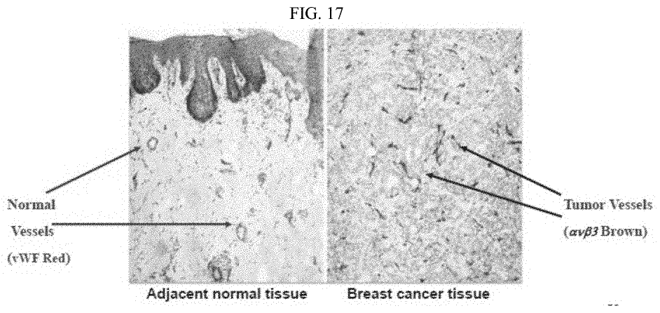

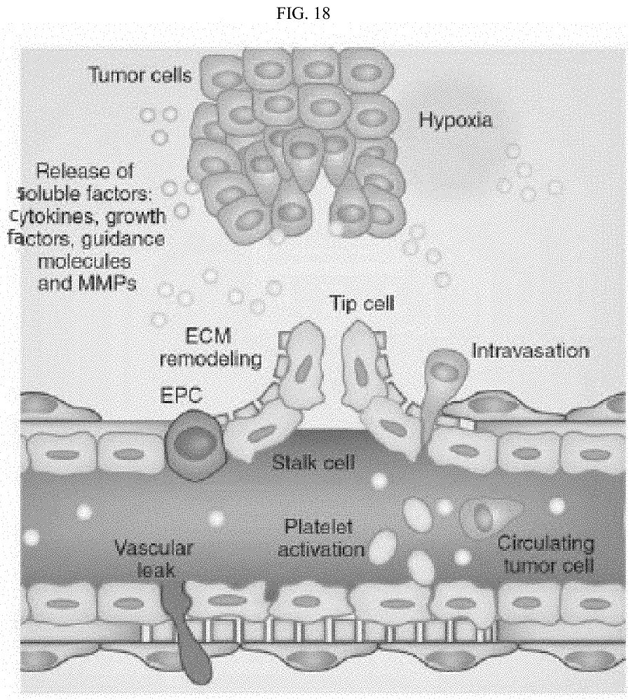

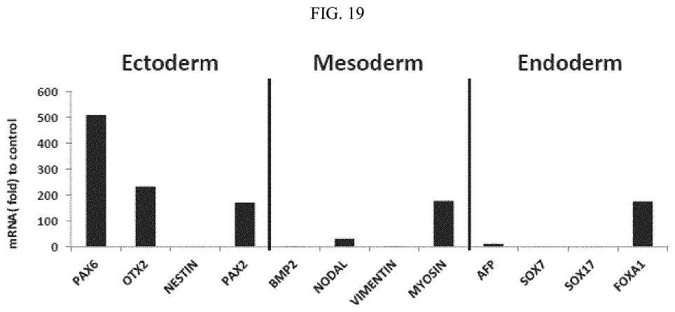

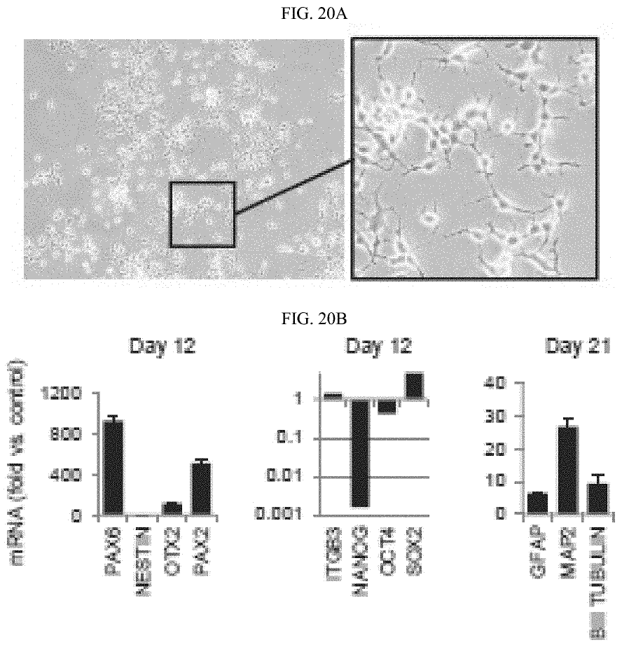

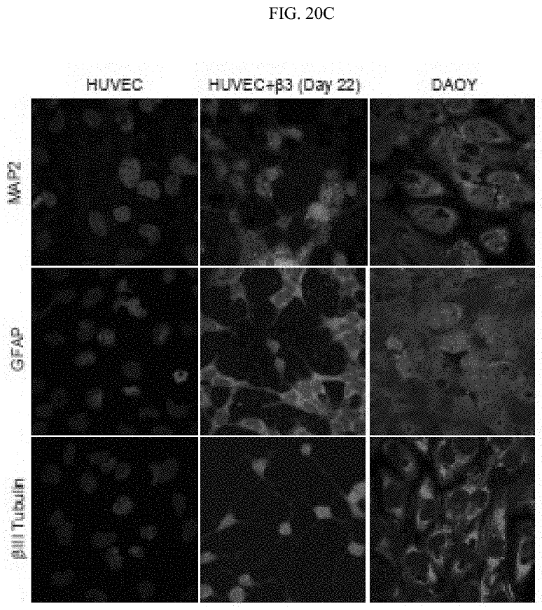

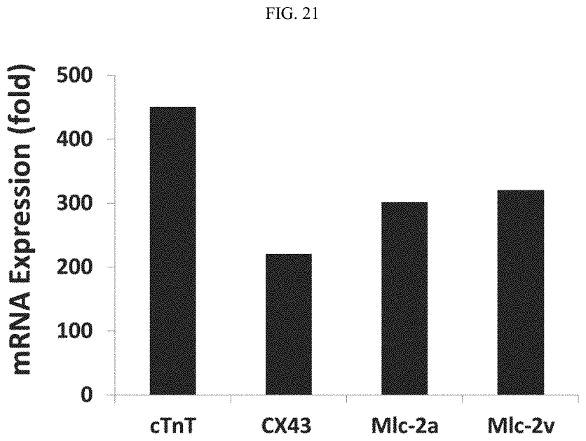

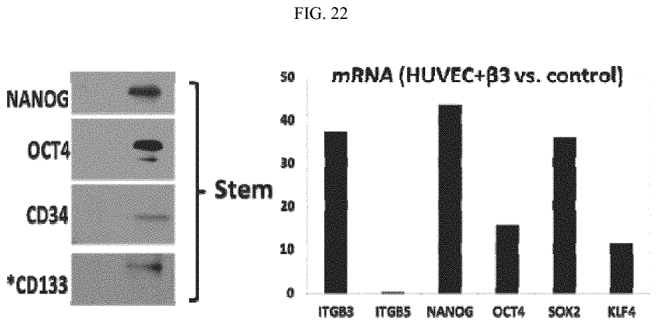

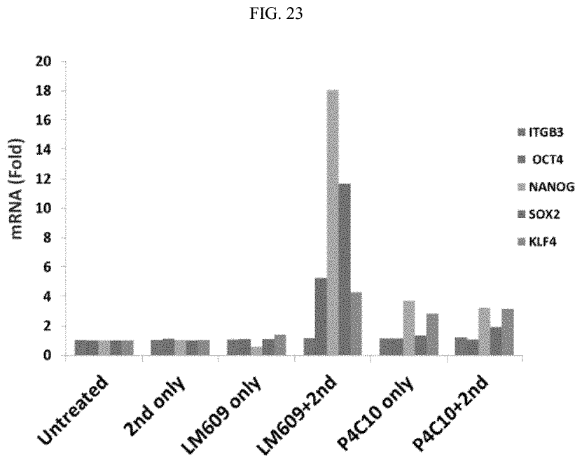

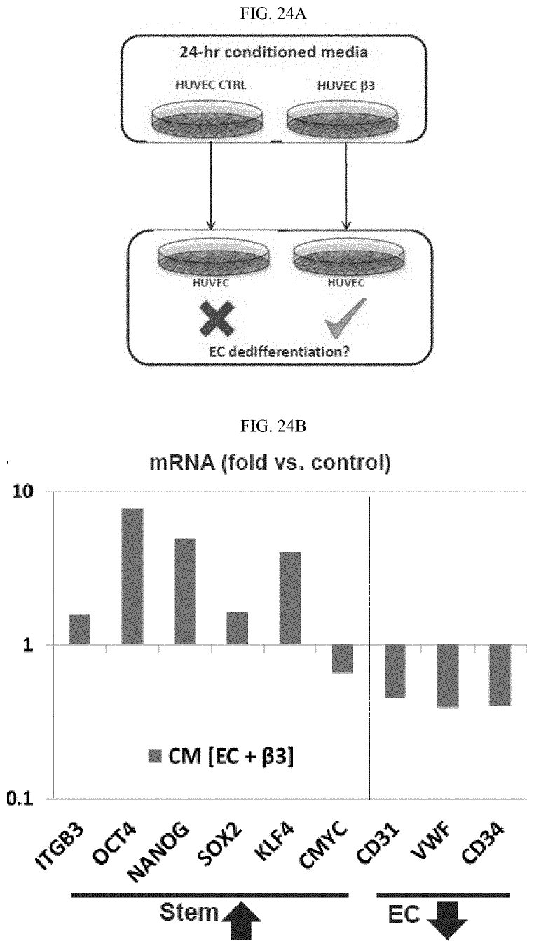

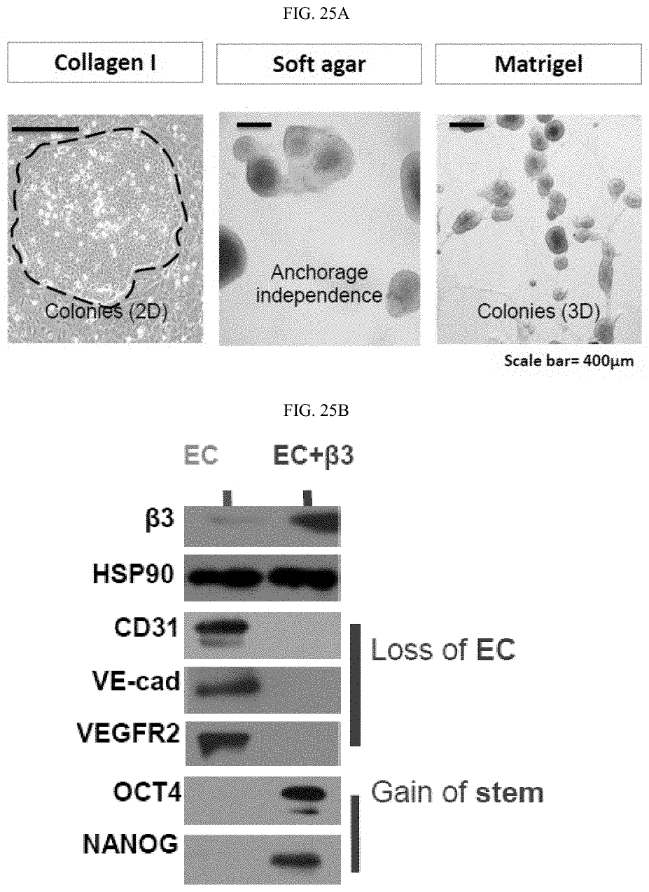

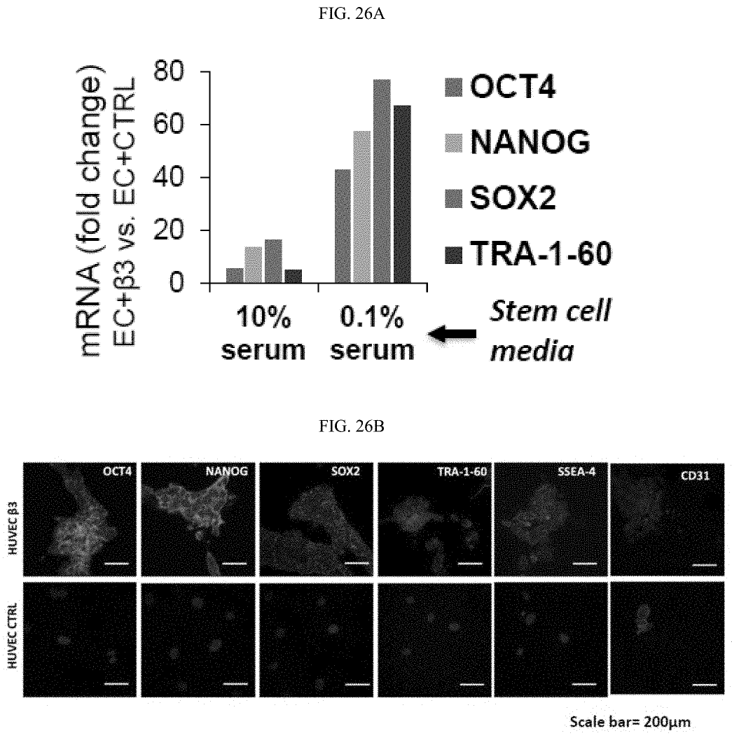

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, provided are methods for making highly dedifferentiated and stem-like human cells from human umbilical vein endothelial cells (HUVECs) ectopically expressing integrin β3 (or ITGB3, also called CD61). In alternative embodiments, provided are methods for inducing undifferentiation and stemness by increasing expression of or ectopically expressing integrin β3, and for differentiating cells by decreasing expression of integrin β3. In alternative embodiments, provided are methods comprising ectopic expression of integrin β3 to: drive NANOG and OCT4 expression; reprogram and transform endothelial cells (ECs), which then lose endothelial markers and/or gain stem markers; generate IPS-like (induced pluripotent stem cell (iPSC)) colony formation. Ectopic expression of integrin β3 in HUVECs can convert these cells to pluripotent stem cells. In alternative embodiments, provided are methods comprising ectopic expression of integrin β3 followed by further differentiating the cells to a particular lineage, e.g., to a neural-ectodermal fate, e.g., differentiation to a neuron. While the invention is not limited by any particular mechanism of action, re-programming by ectopic expression of integrin β3 may be driven by a RAS complex. In alternative embodiments, the integrin β3 is ectopically expressing in a cell by use of a vector, e.g., a lentiviral vector.

Also provided are methods for making ectoderm, mesoderm, and endoderm cells from HUVECs ectopically expressing integrin β3.

Also provided are methods for making neural cells, or cells having neuronal-like morphology, from HUVECs ectopically expressing integrin β3.

Provided are methods for making cardiomyocytes, or cells having cardiomyocyte-like morphology, from HUVECs ectopically expressing integrin β3.

Provided are methods for the production of pluripotent or multipotent stem cells comprising expressing integrin β3 (or b3) in primary human endothelial cells. In alternative embodiments, provided are methods using a single gene, integrin β3, to reprogram somatic cells.

In alternative embodiments, provided are methods for reprogramming endothelial cells into a dedifferentiated state and creating an induced pluripotent stem cell (iPSCs) by inducing αvβ3 clustering. In alternative embodiments, provided are methods for promoting or initiating endothelial remodeling by inducing αvβ3 clustering, where the inducing of αvβ3 (avb3) clustering can lead to increased or enhanced tissue remodeling and/or repair following, e.g., myocardial infarction, stroke, diabetic ulcers, injury (e.g., traumatic or surgical) and other ischemic conditions or diseases. In alternative embodiments, provided are methods for αvβ3 (avb3) clustering comprising administration of e.g., multivalent antibodies or other multivalent ligands that bind to can cause clustering of αvβ3, including multivalent peptides that bind to can cause clustering of αvβ3 or αvβ3 polypeptide ligands, lectins or viral coat proteins that bind to can cause clustering of αvβ3, hydrogels or other polymers that can cause clustering of αvβ3, extracellular matrix (ECM) polymers or polypeptides that can cause clustering of αvβ3, and the like. In alternative embodiments, compositions that can cause clustering of αvβ3 used to practice embodiments provided herein include tri-, quad- or penton (5)-comprising (or more) tripeptide Arg-Gly-Asp (RGD) sites capable of binding αvβ3; polymers, e.g., hydrogels or ECM proteins, comprising multi-RGD peptide sites for clustering of αvβ3. In alternative embodiments, compositions that can cause clustering of αvβ3 used to practice embodiments provided herein include polysaccharides, lectins such as galectin-3, and extracellular matrix (ECM) proteins, some of which are involved in wound healing, including e.g., vitronectin, fibrinogen, and fibronectin.

While the invention is not limited by any particular mechanism of action, the clustering of αvβ3 causes stem-like reprogramming of endothelial cells, which increases endothelial cell plasticity during angiogenesis, which leads to increased or enhanced vascularization and tissue modeling and/or repair following, e.g., myocardial infarction, stroke, diabetic ulcers, injury (e.g., traumatic or surgical) and other ischemic conditions or diseases.

We have demonstrated that αvβ3 (avb3) is the endothelial cell receptor that mediates vascular remodeling on intact heart-derived extracellular matrix (ECM) (hydrogel); and that when compositions capable of clustering αvβ3 (avb3) (e.g., compositions that are multivalent ligands, or greater than bivalent ligands, to αvβ3 (avb3)) are injected into the heart, after vascular injury such as a myocardial infarction (MI), this promotes an improved post-MI outcome and results in less heart damage due to increased neovascularization. Accordingly, in alternative embodiments, by promoting αvβ3 (avb3) multivalent clustering with one or more of agents, e.g., by administration of multivalent ligands, or greater than bivalent ligands, to αvβ3 (avb3)), provided are methods for improving or accelerating angiogenesis in a tissue (e.g., the heart, brain) following an ischemic injury, thereby improving tissue remodeling and minimizing long term tissue injury or damage. In alternative embodiments, methods as provided herein are applied (administered) to patients with injuries, stroke or myocardial infarction (MI), e.g., provided herein are methods for improving tissue remodeling and/or minimizing tissue injury or damage due to e.g., strokes, MIs or other tissue injuries causing ischemia or ameliorated by improving tissue remodeling and accelerating vascularization. In alternative embodiments, methods as provided herein are applied (administered) to individuals, e.g., patients, to enhance or accelerate wound healing.

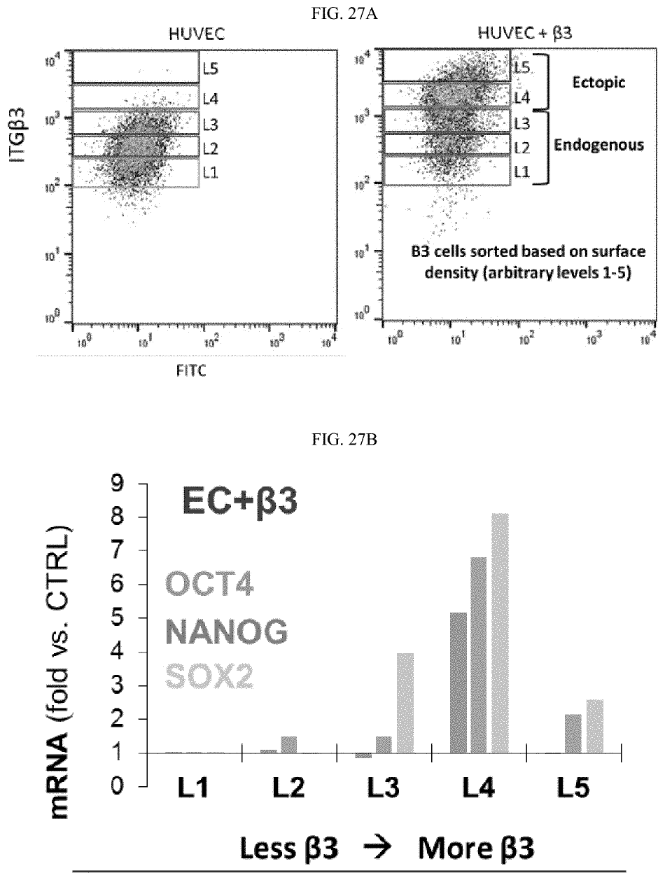

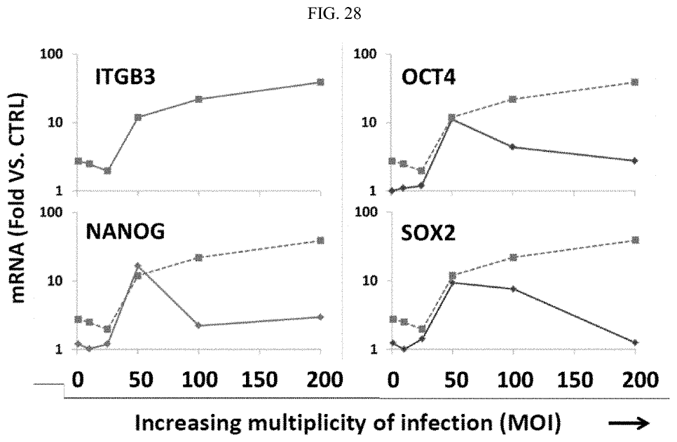

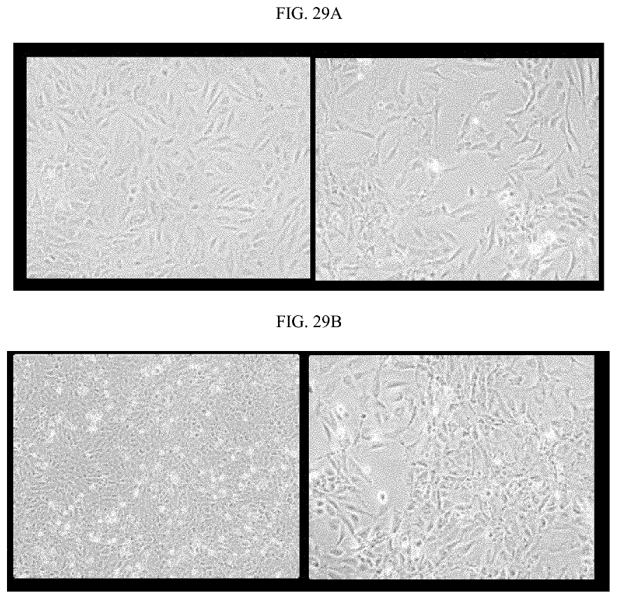

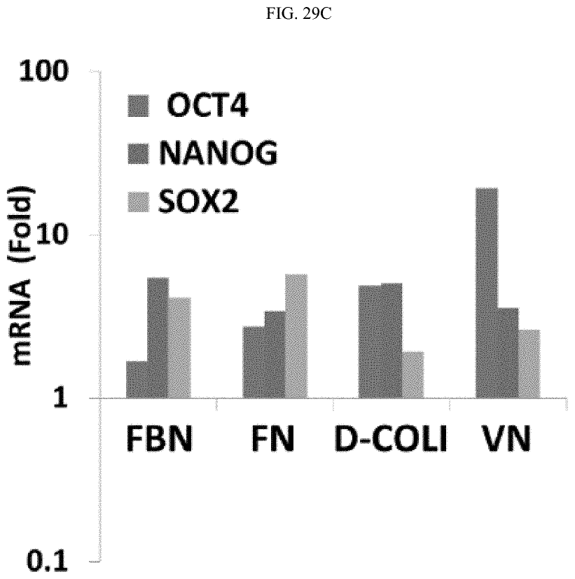

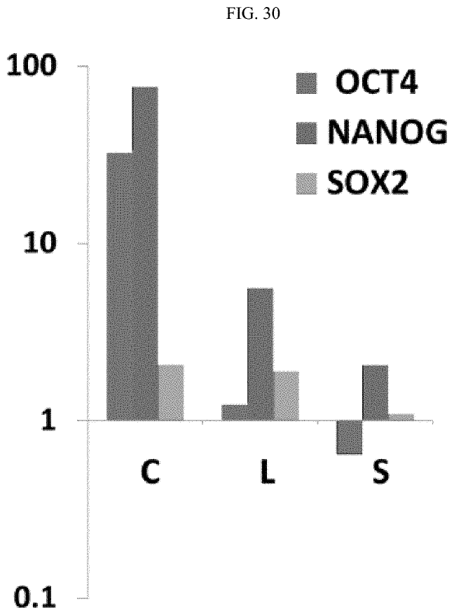

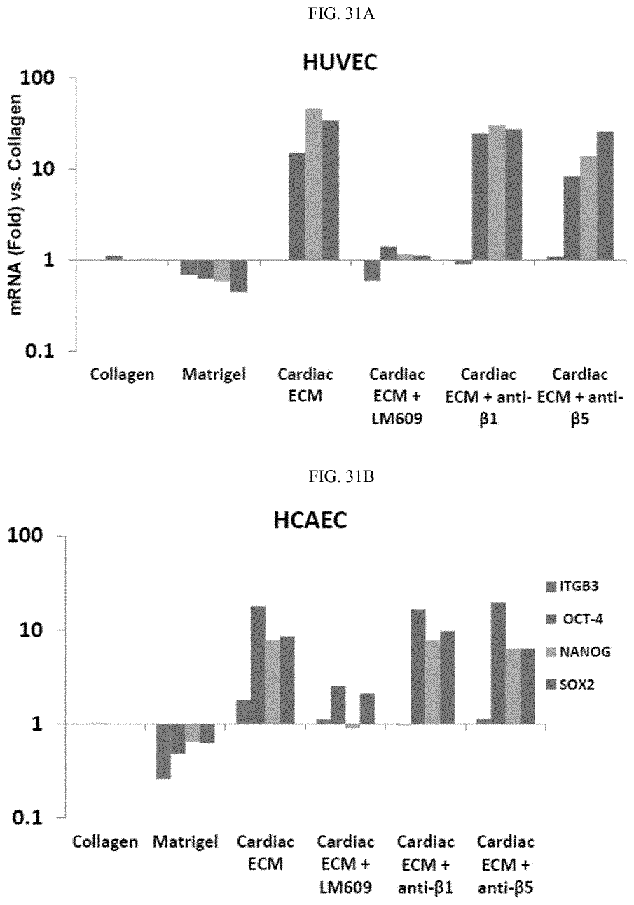

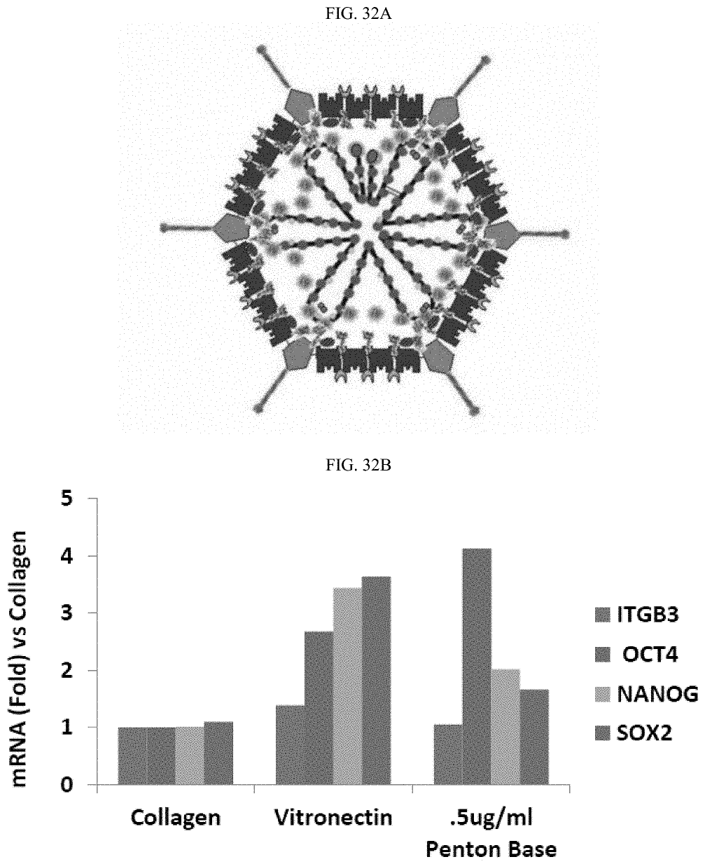

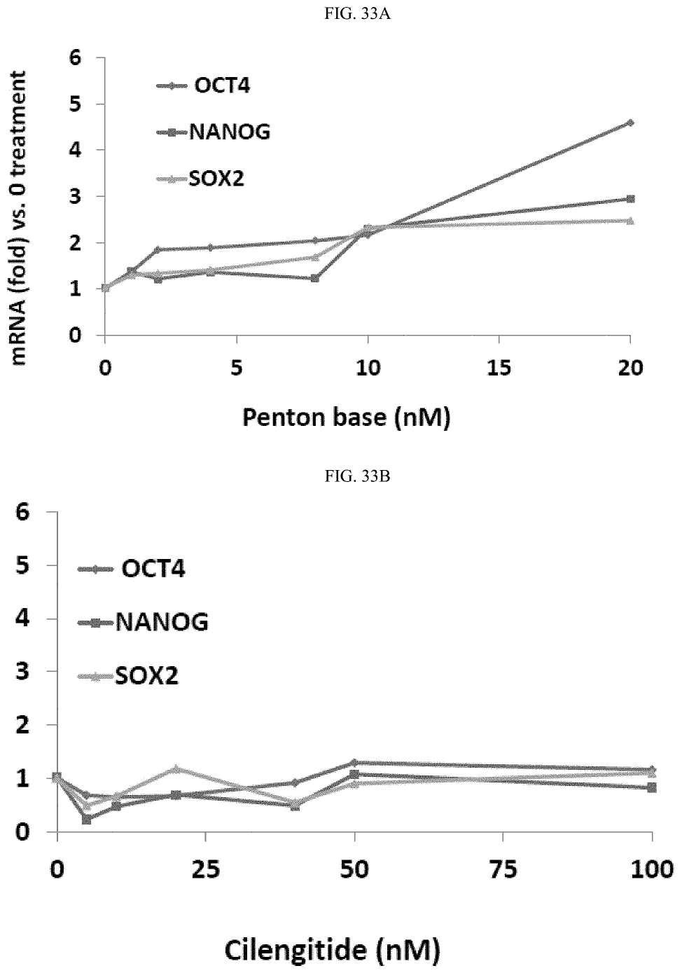

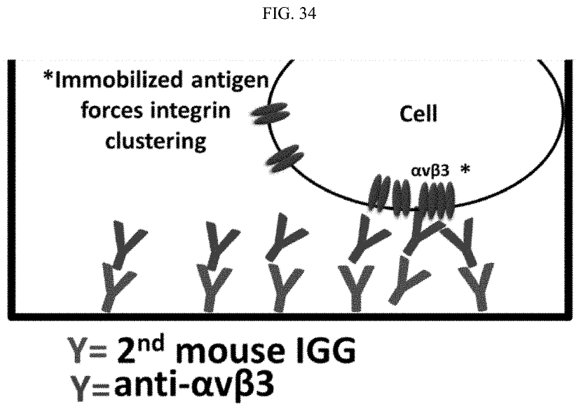

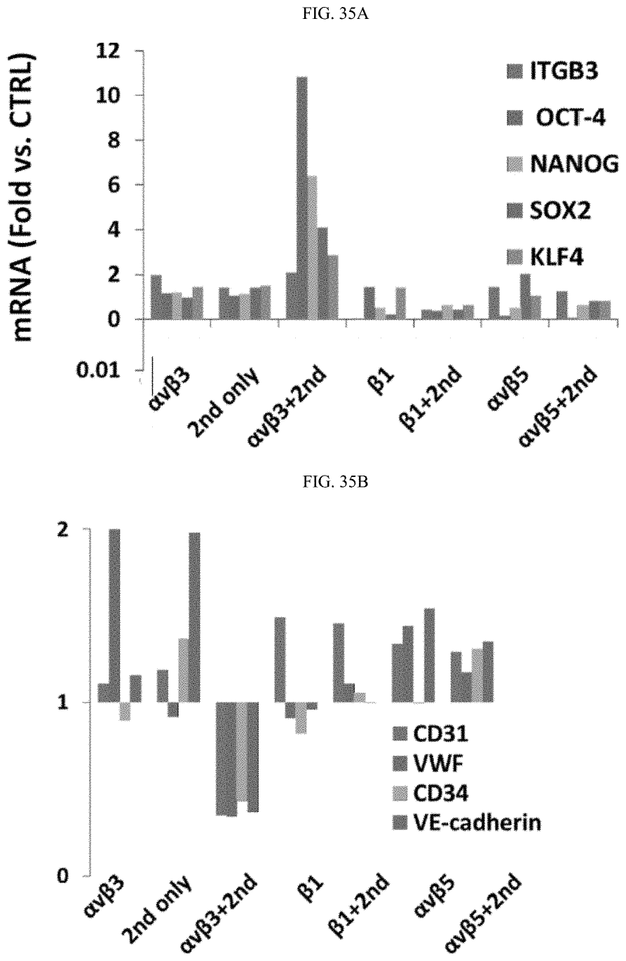

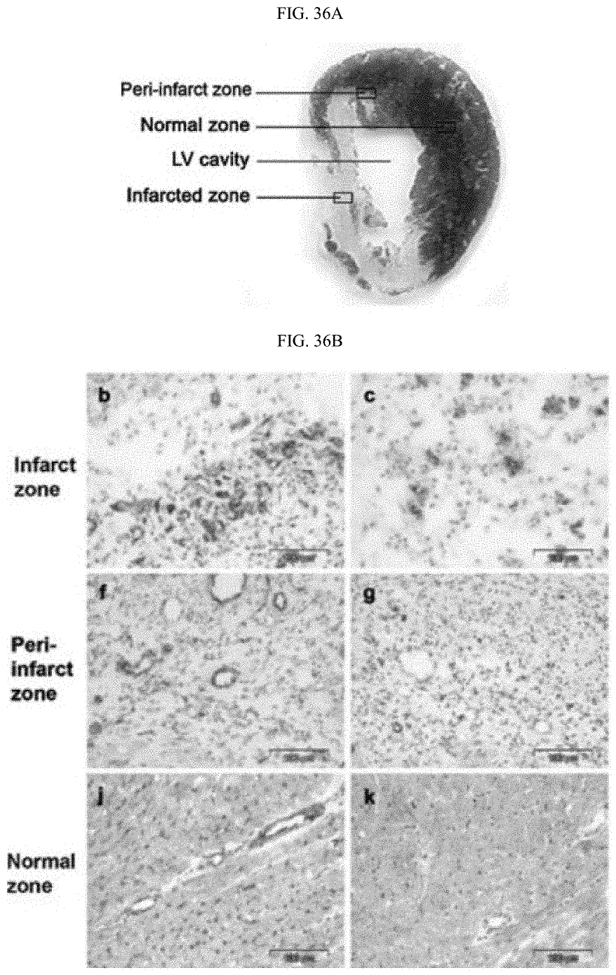

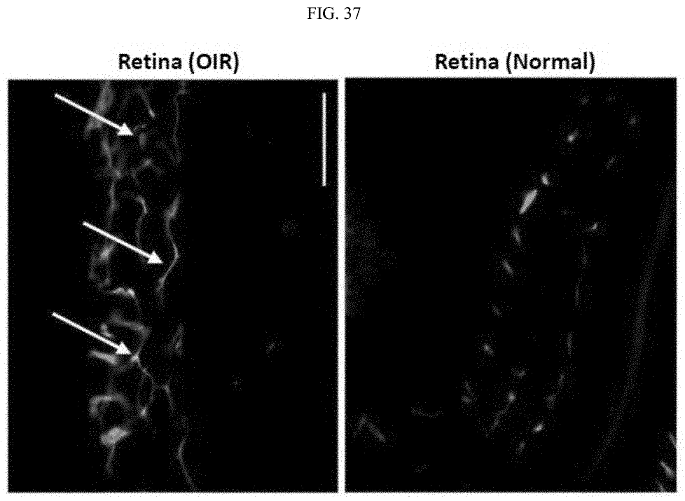

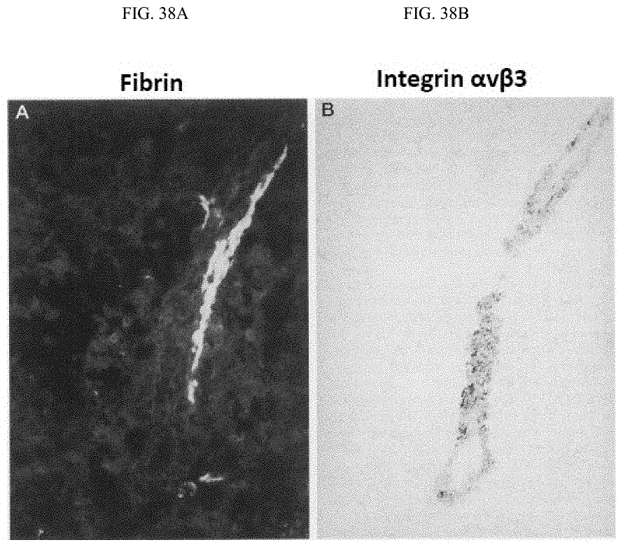

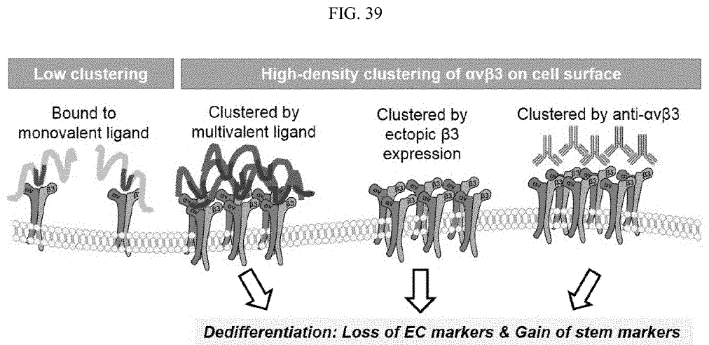

Provided herein are data showing: that a specific amount or level of ectopic integrin β3 expression is required for optimal “clustering” of αvβ3 on the cell surface (see e.g., FIG. 27A-B, FIG. 28); in alternative embodiment, methods provided herein use multivalent ligands (substrates and/or molecules) to cluster integrin αvβ3 on the cell (e.g., an endothelial cell) surface and drive the expression of pluripotent stem genes OCT4, NANOG and SOX2. In alternative embodiments, multivalent (greater than bivalent) ligands are required to cluster αvβ3 on the cell surface. Specific examples include, for example, penton base (5-RGD molecule) and a lectin such as galectin-3; extracellular matrix proteins (ECM) (e.g., as ECMs involved in wound healing) such as vitronectin, fibrinogen, and fibronectin, multivalently bind to αvβ3 and also induce stem gene expression; and, tissue derived-ECM (hydrogels) drive αvβ3-dependent stem gene expression.

In alternative embodiments, methods as provided herein, e.g., for initiating or accelerating tissue regeneration, enhancing or accelerating wound healing, improving tissue remodeling and/or minimizing tissue injury or damage due to e.g., strokes, MIs or other tissue injuries causing ischemia or ameliorated by improving or enhancing tissue remodeling and accelerating vascularization comprise use of ECMs such as tissue-derived ECMs, ECM-derived hydrogels or equivalents, e.g., using decellularized myocardial matrix hydrogels, e.g., from an individual or an animal, e.g., from a human or a porcine source. In alternative embodiments, the decellularized myocardial matrix hydrogels create a microenvironment for cardiac regeneration; in vivo experiments have demonstrated improved ventricular function, increased cardiac muscle, and cellular recruitment after myocardial infarction.

In alternative embodiments, a decellularized myocardial matrix hydrogel used to practice embodiments as provided herein comprises isolation of animal or human heart tissue, generating decellularized myocardium, lyophilizing and milling extracellular matrix material, and then creating a hydrogel; e.g., as described in Wang et al (2016, Jan. 15) Adv Drug Deliv Rev.; vol 96:77-82.

In alternative embodiments, provided are methods for reprogramming and dedifferentiating normal HUVEC to a pluripotent state by exposing or incubating these cells with conditioned media from HUVEC ectopically expressing integrin 133.

### Extracellular Matrix Materials (ECMs)

In alternative embodiments, provided are methods comprising administration to an individual in need thereof a multivalent composition capable of clustering αvβ3 on a cell, e.g., an endothelial cell, surface, wherein exemplary multivalent compositions comprise Extracellular Matrix Materials (ECMs), including as tissue derived-ECM, decellularized ECMs. In alternative embodiments, ECMs, or ECM-comprising compositions or materials used to practice methods as provided herein comprise individual αvβ3-clustering components of ECMs or mixtures thereof, including e.g., vitronectin, fibrinogen and/or fibronectin, also including vitronectin, fibrinogen or fibronectin or fragments thereof, which can be derived from recombinant, synthetic, or tissue or organ sources.

In alternative embodiments, ECMs used to practice methods as provided herein, or methods for making or using the ECMs, include ECMs and methods as described, e.g., in U.S. Pat. Nos. 9,801,983; 9,801,976 and 9,801,975; 9,801,910 (describing methods of making tissue-derived ECM derived from decellularized tissue); 9,795,713 (describing methods of manufacturing bioactive gels from ECM); 9,789,224 (describing digested, decellularized extracellular matrix derived from cardiac tissue); 9,788,821 (describing modified ECMs); 9,744,265 (describing cardiac fibroblast-derived 3-dimensional ECMs); 9,623,051 and 9,572,911 (describing methods of making decellularized ECMs); 8,691,276 (describing solubilized ECMs useful as cell growth scaffolds); or U.S. Pat App Pub nos: 20170312394 (describing an emulsified or injectable ECM); 20170173217 (describing methods for preparing sterilized, gelled, solubilized ECMs); 20170128624, 20170020927 and 20160279170 (describing methods of making decellularized ECMs); 20160354447 (describing compositions and methods using engineered cardiac fibroblast-derived 3-dimensional ECMs); 20160166735 (describing making injectable ECMs).

### Hydrogels

In alternative embodiments, provided are methods comprising administration to an individual in need thereof a multivalent composition capable of clustering αvβ3 on a cell, e.g., an endothelial cell, surface, wherein exemplary multivalent compositions comprise hydrogels such as tissue derived-ECM hydrogels, decellularized tissue hydrogels, and the like.

In alternative embodiments, any hydrogel capable of clustering αvβ3 on a cell, e.g., an endothelial cell, surface can be used to practice methods as provided herein, including for example, hydrogels complexed with multi-RGD peptides or anti-αvβ3 antibodies.

In alternative embodiments, hydrogels used to practice methods as provided herein, or methods for making or using the hydrogels, include hydrogels and methods as described, e.g., in the publications WO/2014/008400, WO/2015/136370, WO/2015/138514 and WO/2017/120092, describing for example PuraStat™ or PuraMatrix™ hydrogels, and/or U.S. Pat. Nos. 9,831,010; 9,814,779; 9,782,490; 9,763,968; 9,688,741; 9,364,412; 8,546,338; or 7,884,185; or U.S. Pat App Pub nos: 20170333304; 20170327813; 20170326275; 20170312368; 20170307598; 20170304499; 20170281781; 20170274082; 20160280827; 20130338084.

### Antibodies

In alternative embodiments, provided are methods comprising the in vitro or in vivo clustering of cell surface αvβ3 by use of an antibody or antibodies that can bind integrin αvβ3 and cluster αvβ3 on the cell surface, or by use of a first antibody that can bind integrin αvβ3 and a second antibody that can bind to the first antibody such that the antibody binding clusters αvβ3 on the cell surface. In alternative embodiments, the antibody or antigen binding fragment thereof can bind to any portion of the integrin αvβ3 protein, whether it be to integrin αv alone, β3 alone, or to a conformational integrin αvβ3 immunogen.

In alternative embodiments, an antibody for practicing methods as provided herein can comprise a peptide or polypeptide derived from, modeled after or substantially encoded by an immunoglobulin gene or immunoglobulin genes, or fragments thereof, capable of specifically binding an antigen or epitope, e.g., αvβ3, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. In alternative aspects, an antibody for practicing methods as provided herein includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, N Y (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

In alternative embodiments, methods as provided herein use “humanized” antibodies, including forms of non-human (e.g., murine) antibodies that are chimeric antibodies comprising minimal sequence (e.g., the antigen binding fragment) derived from non-human immunoglobulin. In alternative embodiments, humanized antibodies are human immunoglobulins in which residues from a hypervariable region (HVR) of a recipient (e.g., a human antibody sequence) are replaced by residues from a hypervariable region (HVR) of a non-human species (donor antibody) such as mouse, rat, rabbit or nonhuman primate having the desired specificity, affinity, and capacity. In alternative embodiments, framework region (FR) residues of the human immunoglobulin are replaced by corresponding non-human residues to improve antigen binding affinity.

In alternative embodiments, humanized antibodies may comprise residues that are not found in the recipient antibody or the donor antibody. These modifications may be made to improve antibody affinity or functional activity. In alternative embodiments, the humanized antibody can comprise substantially all of at least one, and typically two, variable domains, in which all or substantially all of the hypervariable regions correspond to those of a non-human immunoglobulin and all or substantially all of Ab framework regions are those of a human immunoglobulin sequence.

In alternative embodiments, a humanized antibody used to practice this invention can comprise at least a portion of an immunoglobulin constant region (Fc), typically that of or derived from a human immunoglobulin.

However, in alternative embodiments, completely human antibodies also can be used to practice this invention, including human antibodies comprising amino acid sequence which corresponds to that of an antibody produced by a human. This definition of a human antibody specifically excludes a humanized antibody comprising non-human antigen binding residues.

In alternative embodiments, antibodies used to practice methods as provided herein comprise “affinity matured” antibodies, e.g., antibodies comprising with one or more alterations in one or more hypervariable regions which result in an improvement in the affinity of the antibody for antigen; e.g., αvβ3, compared to a parent antibody which does not possess those alteration(s). In alternative embodiments, antibodies used to practice methods as provided herein are matured antibodies having nanomolar or even picomolar affinities for the target antigen, e.g., αvβ3. Affinity matured antibodies can be produced by procedures known in the art.

### Pharmaceutical Compositions and Formulations

In alternative embodiments, provided are pharmaceutical compositions and formulations for practicing methods as provided herein, e.g., methods for inducing αvβ3 clustering in an individual in need thereof, e.g., to accelerate or facilitate angiogenesis, tissue remodeling or repair, or wound healing, for example, to accelerate healing after an infarction. In alternative embodiments, pharmaceutical compositions and formulations for practicing methods as provided herein comprise, e.g., an antibody or antibodies that can bind integrin αvβ3 and cluster αvβ3 on the cell surface, or a multivalent (greater than bi-valent) compound capable of clustering cell surface αvβ3.

In alternative embodiments, compositions used to practice the methods as provided herein are formulated with a pharmaceutically acceptable carrier. In alternative embodiments, the pharmaceutical compositions used to practice the methods as provided herein can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods as provided herein can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods as provided herein include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods as provided herein can be prepared according to any method known to the art for the manufacture of pharmaceuticals. Such drugs can contain preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Aqueous suspensions can contain an active agent (e.g., a composition used to practice the methods as provided herein) in admixture with excipients suitable for the manufacture of aqueous suspensions. Such excipients include a suspending agent, such as sodium carboxymethylcellulose, methylcellulose, hydroxypropyl-methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In practicing methods provided herein, the pharmaceutical compounds can be delivered by transdermally, by a topical route, formulated as applicator sticks, solutions, suspensions, emulsions, gels, creams, ointments, pastes, jellies, paints, powders, and aerosols.

In practicing methods provided herein, the pharmaceutical compounds can also be delivered as nanoparticles or microspheres for regulated, e.g., fast or slow release in the body. For example, nanoparticles or microspheres can be administered via intradermal injection of the desired composition, which slowly releases subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674. Nanoparticles can also be given intravenously, for example nanoparticles with linkage to biological molecules as address tags could be targeted to specific tissues or organs.

In practicing methods provided herein, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations used to practice the methods as provided herein can be lyophilized. Provided are a stable lyophilized formulation comprising a composition as provided herein, which can be made by lyophilizing a solution comprising a pharmaceutical as provided herein and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations used to practice the methods as provided herein can be delivered by the use of liposomes or nanoliposomes. By using liposomes, particularly where the liposome surface carries ligands specific for target cells, e.g., liver cells, or are otherwise preferentially directed to a specific organ, e.g., liver, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

### Nanoparticles, Nanolipoparticles and Liposomes

Also provided are nanoparticles, nanolipoparticles, vesicles and liposomal membranes comprising compounds used to practice the methods as provided herein, e.g., to deliver compositions used to practice methods as provided herein (e.g., an antibody or antibodies that can bind integrin αvβ3 and cluster αvβ3 on the cell surface, or a multivalent (greater than bi-valent) compound capable of clustering cell surface αvβ3) to mammalian, e.g., heart, brain, skin, tenon or other tissues or organs, in vivo, in vitro or ex vivo. In alternative embodiments, these compositions are designed to target specific molecules, including biologic molecules, such as polypeptides, including cell surface polypeptides, e.g., for targeting a desired cell type, e.g., heart, brain, skin, tenon or other tissues or organs.

Provided are multilayered liposomes comprising compounds used to practice methods as provided herein, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, e.g., as described in Park, et al., U.S. Pat. Pub. No. 20070042031, including method of producing a liposome by encapsulating an active agent (e.g., an antibody or antibodies that can bind integrin αvβ3 and cluster αvβ3 on the cell surface, or a multivalent (greater than bi-valent) compound capable of clustering cell surface αvβ3), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, e.g., for targeting delivery of a compound (e.g., an antibody or antibodies that can bind integrin αvβ3 and cluster αvβ3 on the cell surface, or a multivalent (greater than bi-valent) compound capable of clustering cell surface αvβ3) used to practice methods as provided herein to a desired cell type (e.g., a liver endothelial cell, a liver sinusidal cell, or any liver tissue in need thereof), as described e.g., in U.S. Pat. Pub. No. 20070110798.

Provided are nanoparticles comprising compounds (e.g., an antibody or antibodies that can bind integrin αvβ3 and cluster αvβ3 on the cell surface, or a multivalent (greater than bi-valent) compound capable of clustering cell surface αvβ3) used to practice methods as provided herein in the form of active agent-containing nanoparticles (e.g., a secondary nanoparticle), as described, e.g., in U.S. Pat. Pub. No. 20070077286. In one embodiment, provided are nanoparticles comprising a fat-soluble active agent used to practice a method as provided herein or a fat-solubilized water-soluble active agent to act with a bivalent or trivalent metal salt.

In one embodiment, solid lipid suspensions can be used to formulate and to deliver compositions used to practice methods as provided herein to e.g., mammalian heart, brain, skin, tenon or other tissues or organs in vivo, in vitro or ex vivo, as described, e.g., in U.S. Pat. Pub. No. 20050136121.

### Products of Manufacture and Kits

Provided are products of manufacture and kits for practicing methods as provided herein, e.g., methods for inducing αvβ3 clustering in an individual in need thereof, e.g., to accelerate or facilitate angiogenesis, tissue remodeling or repair, or wound healing, for example, to accelerate healing after an infarction. In alternative embodiment, products of manufacture and kits include instructions for practicing methods as provided herein. In alternative embodiment, products of manufacture and kits comprise compositions for practicing methods as provided herein, e.g., an antibody or antibodies that can bind integrin αvβ3 and cluster αvβ3 on the cell surface, or a multivalent (greater than bi-valent) compound capable of clustering cell surface αvβ3.

Ectopic Expression of Integrin β3 in HUVECs Drives the Up-Regulation of Pluripotency Genes and Converts these Cells into a Highly Dedifferentiated and Stem-Like State

We discovered that ectopic expression of integrin β3 (or ITGB3) in HUVECs drives the up-regulation of several known pluripotency genes (NANOG, OCT4, SOX2, and KLF4), and, based on these finding, provided are methods for making highly dedifferentiated and stem-like human cells by ectopic expression of integrin β3 in human endothelial cells to convert these cells into a highly dedifferentiated and stem-like state. That HUVECs with ectopic β3 are converted to a dedifferentiated state is evidenced by their loss of several distinguishing markers associated with endothelial identity, including CD31, VWF, VE-cadherin, and VEGFR2, and the gain of pluripotency markers such as NANOG, OCT4, SOX2, and KLF4.

Similar to induced pluripotent stem cells, HUVEC with ectopic integrin β3 (or ITGB3) expression underwent both spontaneous and directed differentiation to other cell types. When pluripotent stem cells are placed in spheroid forming conditions, they spontaneously express lineages markers associated with all three germ layers (ectoderm, mesoderm, and endoderm). Growing HUVEC β3 cells under these same conditions produced the spontaneous induction of lineage markers associated with all three germlines. Thus, provided are methods for making ectoderm, mesoderm, and endoderm cells from HUVECs comprising ectopically expressing β3 in HUVECs and growing or incubating the cells in spheroid forming conditions, thereby inducing all three germlines.

To further test whether HUVEC β3 could be directly differentiated toward another cell type, we cultured our cells under neuronal differentiating conditions. By day 12, cells acquired a neuronal-like morphology and began to express early neuro-ectodermal lineage markers, and lost expression of pluripotency genes NANOG and OCT4. Furthermore, by day 22 cells began to express mature neuronal markers. Thus, provided are methods for making neural cells, or cells having neuronal-like morphology, from HUVECs ectopically expressing integrin β3 (or ITGB3).

In a second experiment, we cultured cells in cardiomyocyte differentiating conditions, and by day 14, the cells began to express cardiomyocyte markers. Thus, provided are methods for making cardiomyocytes, or cells having cardiomyocyte-like morphology, from HUVECs ectopically expressing integrin β3 (or ITGB3).

In alternative embodiments, provided are methods for the production of pluripotent or multipotent stem cells comprising expressing integrin β3 (or b3) in primary human endothelial cells, e.g., by using transduction of a vector or a virus or the like, e.g., a lentivirus transduction, upon which the primary endothelial cells become reprogrammed into stem-like cells after 12-15 days. Ectopic expression of ITGB3 reprograms human umbilical vein endothelial cells (HUVEC) into a dedifferentiated stem cell-like state. This is characterized by the expression of pluripotency genes NANOG, OCT4, SOX2, KLF4 and a loss of endothelial marker expression (CD31, VWF, VE-Cadherin, and VEGFR2). These cells have been demonstrated to differentiate into neuronal and cardiomyocyte cells.

In alternative embodiments, provided are methods improving on a standardized approach to reprogram somatic cells requiring the introduction of 4 genes known as the Yamanaka factors (Oct-4, Sox-2, Klf4 and c-Myc) into target cells using retroviruses; following stable introduction of the Yamanaka factors into somatic cells (6 days), cellular reprogramming to induced pluripotent stem cells (iPSCs) takes approximately 30 days. Provided herein are methods for reprogramming somatic cells, e.g., reprogramming endothelial cells, with the introduction of a single gene, ITGB3 and which in some embodiments can be done in a shorter period of time, for example, between about 12 to 20 days versus (vs.) 30 days. Provided are data demonstrating that ITGB3 strongly induces Oct-4, Sox-2 and Klf4, three of the four Yamanaka factors, and this observation supports this invention's finding that high levels of ITGB3, or increasing integrin β3 (or ITGB3) in a cell, are sufficient to reprogram endothelial cells.

Additionally, the cultivation of iPSCs requires expensive cell growth media and feeder cells, and in terms of costs, an alternative embodiment provided herein requires less expensive cell growth media, e.g., standard endothelial vs. stem cell media, such that costs are reduced by approximately 2 fold. Provided herein are methods comprising the ectopic expression of one gene ITGB3 (versus the overexpression of four genes Oct-4, Sox-2, Klf4 and c-Myc) to reprogram endothelial cells. In summary, in alternative embodiments, provided are methods requiring less time and expenses with the use of a single gene to reprogram somatic cells.

Finding that ITGB3 strongly induces Oct-4, Sox-2, Nanog and Klf4, all potent reprogramming factors, we discovered that a high threshold level of ITGB3 facilitates reprogramming in endothelial cells. We have documented clustering and induction of activated Ras by ITGB3 overexpression in endothelial cells. Downstream of Ras, we observed a strong activation of Akt and the suppression of activated extracellular signal-regulated kinase (ERK). It has been reported that ERK activation impairs cellular reprogramming while Akt promotes it. Akt is known to phosphorylate and stabilize Oct-4. Once stabilized, Oct-4 then cooperates with Sox-2 to maintain a stem phenotype by inducing Nanog. We found that high ITGB3 levels facilitate the clustering of Ras family members, that then activates Akt to stabilize the pluripotency regulator Oct-4.

In alternative embodiments, provided are methods that are a more effective and efficient means for the generation of pluripotent or multipotent stem cells. In alternative embodiments, patient-derived primary cells are used to create personalized therapeutics, to test drug responsiveness on patient-derived cells such as neurons or cardiomyocytes, or for transplantation.

Clustering of Integrin αvβ3 Drives the Up-Regulation of Pluripotency Genes, Reprogram Endothelial Cells into a Dedifferentiated State, and Creates an Induced Pluripotent Stem Cell

We have discovered that clustering of integrin αvβ3 (through an integrin cross-linking assay) drives the up-regulation of several known pluripotency genes (NANOG, OCT4, SOX2, and KLF4) in Human Umbilical Vein Endothelial Cells (HUVEC) (see figure), and that αvβ3 clustering alone is sufficient to reprogram endothelial cells into a dedifferentiated state, and create an induced pluripotent stem cell (iPSCs). In alternative embodiments, clustering of αvβ3 yields (makes, generates) iPSCs with similar biologic properties to what is achieved by current technologies that drive overexpression of pluripotency genes (OCT-4, SOX2, KLF4, and C-myc). In alternative embodiments, provided are methods that circumvent the known caveats of malignant transformation associated with current gene transduction technologies.

In alternative embodiments, these processes can be facilitated by any multivalent ligand that binds to either integrin αv or β3, and clusters them on the cell surface. In alternative embodiments, several ways of achieving this are using: an antibody integrin cross-linking assay (both adherent and in suspension); a pentavalent molecule such as a lectin, e.g., Galectin-3, to cluster αvβ3; a mimetic RGD peptide (αvβ3 binding motif) to bind and cluster αvβ3; manganese cations (Mn2+), which are known to activate and cluster αvβ3.

In alternative embodiments, provided are methods for the production of pluripotent or multipotent stem cells from readily accessible sources of HUVEC and/or adult somatic cells. Briefly, while the invention is not limited by any particular mechanism of action, αvβ3 integrin clustering drives the up-regulation of pluripotent stem genes (NANOG, OCT4, SOX2, KLF4) which will reprogram somatic cells into a dedifferentiated state. From this state, provided are methods that differentiate cells using appropriate conditions. For example, provided are methods to differentiate HUVECs to neurons and cardiomyocytes.

In alternative embodiments, provided are methods that improve on known standardized approaches to reprogram somatic cells which require the introduction of 4 genes known as the Yamanaka factors (Oct-4, Sox-2, Klf4 and c-Myc) into target cells using retroviruses; where following stable introduction of the Yamanaka factors into somatic cells (6 days), cellular reprogramming to induced pluripotent stem cells (iPSCs) takes approximately 30 days. In alternative embodiments, provided are methods that can reprogram endothelial cells with the simple manipulation of an integrin αvβ3 on the cell surface.

Current methods for stem cell reprogramming require some form of genetic manipulation, and this comes with many caveats, including genetic instability and the possibility of tumor formation. In alternative embodiments, provided are methods that completely αvoid this possibility. Current methods for the cultivation of iPSCs require expensive cell growth media and feeder cells. In alternative embodiments, provided are methods that require less expensive cell growth media (standard endothelial vs stem cell media) such that costs are reduced by approximately 2 fold. In summary, in alternative embodiments, provided are methods for the creation of pluripotent stem cells that involved no genetic manipulation or special and expensive media conditions.

In alternative embodiments, provided are methods that cluster avb3 (versus genetic manipulation by over expressing four genes (oct-4, Sox-2, Klf4 and c-Myc) used by all current methods). In alternative embodiments, provided are methods that can create stem cells that do not require genetic manipulation, which has many potential adverse side effects. In alternative embodiments, provided are methods comprising only temporary clustering of avb3 on the cell surface; this exemplary process can αvoid all the pitfalls associated with the currents methods for stem cell production. In alternative embodiments, provided are methods that are more effective and efficient way for the generation of pluripotent or multipotent stem cells. In alternative embodiments, provided are methods for using patient-derived primary cells to create personalized therapeutics, to test drug responsiveness on patient-derived cells such as neurons or cardiomyocytes, or for transplantation.

Conditioned Media from HUVEC Ectopically Expressing Integrin 133 is Capable of Reprogramming and Dedifferentiating Normal HUVEC to a Pluripotent State

We have discovered that Human umbilical vein endothelial cells (HUVEC) ectopically expressing integrin 133 (HUVEC 133⁺) conditions its media. This altered or conditioned media (CM), when cultured with normal HUVEC, is capable of reprogramming and dedifferentiating these cells to a pluripotent state, resulting in the loss of endothelial markers (CD31, VWF, and CD34), and the gain pluripotency gene expression (OCT4, NANOG, SOX2, KLF4) (see attached figure). HUVEC 133 CM alone is sufficient to reprogram endothelial cells into a dedifferentiated state creating an induced pluripotent stem cell (iPSCs). In alternative embodiments, provided are methods for making and using HUVEC 133 CM to yield iPSCs having similar biological properties achieved with conventional stem cell technologies that drive overexpression of pluripotency genes (OCT4, SOX2, KLF4, and C-MYC).

In alternative embodiments, provided are methods that require no genetic manipulation of our target cells, circumventing the known caveats of malignant transformation associated with current gene transduction technologies.

In alternative embodiments, provided are methods for the production of pluripotent or multipotent stem cells from readily accessible sources of Human Umbilical Vein Endothelial Cells (HUVEC). In alternative embodiments, provided are methods comprising collection of conditioned media (CM) from HUVEC ectopically expressing integrin 133, and culturing them with normal HUVEC to drive the up-regulation of pluripotent stem genes (NANOG, OCT4, SOX2, KLF4), and loss of endothelial markers (CD31, VWF, and CD34). In alternative embodiments, this treatment reprograms HUVEC into a dedifferentiated, pluripotent state, and these cells can then be differentiated using appropriate conditions; for example, dedifferentiated HUVECs can be induced to differentiate to neurons and cardiomyocytes.

In alternative embodiments, provided are methods that improve on the standardized approach to reprogram somatic cells which requires the introduction of 4 genes known as the Yamanaka factors (Oct-4, Sox-2, Klf4 and c-Myc) into target cells using retroviruses, and following stable introduction of the Yamanaka factors into somatic cells (6 days), cellular reprogramming to induced pluripotent stem cells (iPSCs) takes approximately 30 days. In alternative embodiments, provided are methods that can reprogram endothelial cells with a simple exposure to conditioned media. All other methods for stem cell reprogramming require some form of genetic manipulation, which comes with many caveats, including genetic instability and the possibility of tumor formation. In alternative embodiments, provided are methods that completely αvoid this possibility.

Known methods for the cultivation of iPSCs require expensive cell growth media and feeder cells. In alternative embodiments, provided are methods requiring less expensive cell growth media (standard endothelial vs stem cell media) such that costs are reduced by approximately 2 fold. In summary, in alternative embodiments, provided are methods for the creation of pluripotent stem cells that involve no genetic manipulation or special and expensive media conditions. In alternative embodiments, provided are methods that involve exposing HUVEC to conditioned media versus genetic manipulation by over expressing four genes (oct-4, Sox-2, Klf4 and c-Myc), as used by all current methods.

While the invention is not limited by any particular mechanism of action, given that conditioned media actively induces the expression of pluripotency genes Oct-4, Nanog, Sox-2, and Klf-4, all potent reprogramming factors, conditioned media from HUVEC 3 cells facilitates endothelial reprogramming. In alternative embodiments, provided are methods that involve exposing HUVEC to conditioned media for a limited time. In alternative embodiments, provided are methods that are an effective and efficient way to generate pluripotent or multipotent stem cells. In alternative embodiments, provided are methods using patient-derived primary cells to create personalized therapeutics, to test drug responsiveness on patient-derived cells such as neurons or cardiomyocytes, or for transplantation.

A number of embodiments of the invention have been described. Nevertheless, it will be understood that various modifications may be made without departing from the spirit and scope of the invention. Accordingly, other embodiments are within the scope of the following claims.

1. A method for:
upregulating pluripotency genes in a somatic cell, or a human endothelial cell,
dedifferentiating the somatic cell or human endothelial cell to become a stem-like cell, or reprogramming the somatic cell or endothelial cell into a dedifferentiated state to generate an induced pluripotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair, and/or
enhancing wound repair,
comprising ectopically expressing integrin β3 in the somatic cell or the human endothelial cell, or expressing or overexpressing in the somatic cell or the human endothelial cell a heterologous integrin β3,
wherein optionally the human endothelial cell is a human umbilical vein endothelial cell (HUVEC),
and optionally the pluripotency gene is a NANOG, OCT4, SOX2, and/or KLF4 gene,
and optionally the dedifferentiated or reprogrammed human endothelial cell loses its endothelial identity, optionally comprising loss of CD31, VWF, VE-cadherin, and VEGFR2, and gain of pluripotency markers such as NANOG, OCT4, SOX2, and KLF4,
and optionally ectopic expression of the integrin β3 in the somatic cell or the human endothelial cell is by transduction of a vector or a virus, optionally an adenovirus or a lentivirus (having contained therein an integrin β3-expressing nucleic acid), and expressing the integrin β3.

2. The method of claim 1, further comprising inducing the dedifferentiated or reprogrammed human somatic or endothelial cell, or the induced pluripotent stem cell (iPSC), to express lineages markers associated with all three germ layers ectoderm, mesoderm and endoderm, and/or inducing the dedifferentiated human somatic or endothelial cell to differentiate to one or all three germ layers ectoderm, mesoderm and endoderm, optionally comprising placing or incubating the dedifferentiated human somatic or endothelial cell in spheroid forming conditions.

3. A method for:
upregulating pluripotency genes in an αvβ3-expressing human somatic cell, an αvβ3-endothelial cell, or an αvβ3-expressing human endothelial cell,
dedifferentiating an αvβ3-expressing human somatic cell, an αvβ3-endothelial cell, or an αvβ3-expressing human endothelial cell to become a stem-like cell,
reprogramming an αvβ3-expressing human somatic cell, an αvβ3-endothelial cell, or an αvβ3-expressing human endothelial cell into a dedifferentiated state to generate an induced pluripotent or multipotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair,
enhancing wound repair, and/or
enhancing or accelerating healing, tissue healing or remodeling, vascularization or revascularization and/or tissue repair after an ischemic event, a tissue injury, a wound, a burn, or an infarction,
wherein optionally the wound is a surgical or traumatic wound,
wherein optionally the infarction is a myocardial infarction (MI) or a brain infarction or a stroke,
and optionally the ischemic event or injury is caused by an occlusion, an embolism or a trauma, or an aneurysm,
and optionally the ischemic event, wound or tissue injury is or is caused by: a diabetic ulcer, a corneal ischemic event, a stroke, a myocardial infarction, a mitral valve disease, a chronic atrial fibrillation, a cardiomyopathy, a prosthesis,
and optionally the tissue healing or remodeling comprises healing or remodeling of skin, connective tissue, fascia, bone, cartilage, tendon or muscle,
and optionally the tissue healing or remodeling comprises healing or remodeling of epidermal or dermal tissues,
and optionally the vascularization or revascularization, tissue healing or remodeling comprises healing or remodeling or the treatment of: retinal ischemia, diabetic retinopathy, or ocular ischemic syndrome (OIS), cardiac ischemia, bowel ischemia or ischemic colitis, brain ischemia, limb ischemia, or cutaneous ischemia, hypotension, sickle cell disease, arteriovenous malformations or peripheral artery occlusive disease,
and optionally the tissue healing or remodeling comprises healing or remodeling trauma or injury due to radiotherapy,
comprising: clustering of cell surface αvβ3,
and optionally the clustering of cell surface αvβ3 is by use of a multivalent ligand that binds to either integrin αv, integrin β3 or integrin αvβ3 (exposure of the cell surface to the multivalent ligand),
and optionally the clustering of cell surface αvβ3 is by (comprises) use of, or the clustering of cell surface αvβ3 comprises administration to an individual in need thereof:
an antibody or antibodies that can bind integrin αvβ3 and cluster αvβ3 on the cell surface, or a first antibody that can bind integrin αvβ3 and a second antibody that can bind to the first antibody such that the antibody binding clusters αvβ3 on the cell surface;
a multivalent compound capable of clustering cell surface αvβ3, wherein optionally the multivalent compound is a pentavalent molecule,
and optionally the multivalent compound capable of clustering αvβ3 on a cell surface comprises:
(a) an extracellular matrix (ECM) protein, or an ECM homogenate or ECM-derived composition, capable of clustering αvβ3 on a cell surface,
and optionally the ECM comprises vitronectin, fibrinogen, and/or fibronectin,
and optionally the ECM comprises a decellularized ECM matrix or an ECM matrix hydrogel, optionally a myocardial matrix or a myocardial matrix hydrogel,
(b) a polysaccharide or glycosylated polypeptide capable of clustering αvβ3 on a cell surface,
(c) a lectin, a lectin capable of specifically binding of β-galactosides, or a Galectin-3 or a Galectin-9, capable of clustering αvβ3 on a cell surface;
(d) a compound comprising three or more RGD peptides or mimetic RGD peptides capable of clustering αvβ3 on a cell surface, wherein optionally the compound comprises a polypeptide or a hydrogel;
(e) manganese cations (Mn2+) or a composition comprising a plurality of manganese cations (Mn2+), capable of clustering αvβ3 on a cell surface;
(f) a viral coat protein, or a composition comprising a plurality of viral coat proteins, a capsid or a virion that can cluster cell surface αvβ3,
(g) any combination of (a) to (f), or equivalents thereof,
thereby:
upregulating pluripotency genes in an αvβ3-expressing human somatic cell, an αvβ3-endothelial cell, or an αvβ3-expressing human endothelial cell,
dedifferentiating an αvβ3-expressing human somatic cell, an αvβ3-endothelial cell, or an αvβ3-expressing human endothelial cell to become a stem-like cell,
reprogramming an αvβ3-expressing human somatic cell, an αvβ3-endothelial cell, or an αvβ3-expressing human endothelial cell into a dedifferentiated state to generate an induced pluripotent or multipotent stem cell (iPSC),
increasing endothelial cell plasticity during angiogenesis,
increasing or enhancing vascularization and/or angiogenesis,
promoting or initiating endothelial remodeling,
increasing, facilitating or enhancing tissue remodeling or repair,
enhancing wound repair, and/or
enhancing or accelerating healing, tissue healing or remodeling, vascularization and/or tissue repair after an ischemic event, a tissue injury, a wound, a burn, or an infarction.

4. The method of claim 3, wherein the antibody or antibodies that can bind integrin αvβ3 and cluster αvβ3 on the cell surface, or the multivalent compound capable of clustering cell surface αvβ3, is:
formulated as a pharmaceutical composition,
formulated with a pharmaceutically acceptable excipient,
administered directly to, into, locally to, or adjacent to, a wound or injury site or tissue, a site or tissue requiring increased or enhanced vascularization and/or angiogenesis, a site or tissue needing promotion or initiation of endothelial remodeling, an infarction site, to an injured or infarcted heart or other tissue or organ, or to any tissue or organ in need of increased or enhanced vascularization or tissue repair,
wherein optionally the administration is by injection or by placement of an implant.

5. The method of claim 3, further comprising inducing the dedifferentiated or reprogrammed human somatic or endothelial cell, or the induced pluripotent stem cell (iPSC), to express lineages markers associated with all three germ layers ectoderm, mesoderm and endoderm, and/or inducing the dedifferentiated human somatic or endothelial cell to differentiate to one or all three germ layers ectoderm, mesoderm and endoderm, optionally comprising placing or incubating the dedifferentiated human somatic or endothelial cell in spheroid forming conditions.

6. A method for:
upregulating pluripotency genes in a αvβ3-expressing human somatic cell or human endothelial cell, and/or
dedifferentiating the αvβ3-expressing human somatic cell or human endothelial cell to become a stem-like cell, or reprogramming the human somatic cell or endothelial cell into a dedifferentiated state to generate an induced pluripotent stem cell (iPSC),
comprising ectopically expressing integrin 133 (HUVEC 133+) or expressing in the human somatic cell or the human endothelial cell a heterologous integrin 133 (HUVEC 133+) to generate a conditioned or altered media, and culturing or exposing the human somatic cell or the human endothelial cell to the conditioned or altered media,
wherein optionally the human endothelial cell is a human umbilical vein endothelial cell (HUVEC),
and optionally the pluripotency gene is a NANOG, OCT4, SOX2, and/or KLF4 gene,
and optionally the dedifferentiated or reprogrammed human endothelial cell loses its endothelial identity, optionally comprising loss of CD31, VWF, VE-cadherin, and VEGFR2, and gain of pluripotency markers such as NANOG, OCT4, SOX2, and KLF4,
and optionally ectopic expression of the integrin 133 (HUVEC 133+) in the human somatic cell or the human endothelial cell is by transduction of a vector or a virus, optionally a lentivirus, expressing the integrin β3 expressing nucleic acid).

7. The method of claim 6, further comprising inducing the dedifferentiated or reprogrammed human somatic or endothelial cell, or the induced pluripotent stem cell (iPSC), to express lineages markers associated with all three germ layers ectoderm, mesoderm and endoderm, and/or inducing the dedifferentiated human somatic or endothelial cell to differentiate to one or all three germ layers ectoderm, mesoderm and endoderm, optionally comprising placing or incubating the dedifferentiated human somatic or endothelial cell in spheroid forming conditions.

8-11. (canceled)
